(19) United States
(12) Patent Application Publication
(10) Pub. No.: US 2012/0077854 A1
(43) Pub. Date: Mar. 29, 2012

(54) COMPOSITIONS AND METHODS FOR MODULATING RETINOL BINDING TO RETINOL BINDING PROTEIN 4 (RBP4)

(75) Inventors: Hank Michael James Petrassi, Cardiff, CA (US); David C. Tully, San Diego, CA (US); Brian T. Masick, Encinitas, CA (US); Bao Nguyen, San Diego, CA (US)

(73) Assignee: IRM LLC, Hamilton (BM)

(21) Appl. No.: 13/258,341

(22) PCT Filed: Apr. 13, 2010
(86) PCT No.: PCT/US10/30843

§ 371 Date: Dec. 12, 2011

Related U.S. Application Data
(60) Provisional application No. 61/168,720, filed on Apr. 13, 2009.

(51) Int. Cl. A61K 31/426; C07D 271/06; C07D 263/32; A61P 27/02; C12N 5/071; A61K 31/4245; A61K 31/421; A61K 31/427; C07D 277/593; C07D 417/06

(52) U.S. Cl. 514/364; 548/204; 548/131; 548/236; 548/202; 435/375; 514/365; 514/374

(57) ABSTRACT

The present invention relates to compositions and methods for modulating retinol binding to retinol binding protein 4 (RBP4). In particular, the present invention provides compounds having Formula (1) or (2) (Formulae (1), (2)); wherein R¹, R², R³, R⁴, R⁵, R⁶, Y¹, Y², Y³, Y⁴ and m are as defined above.

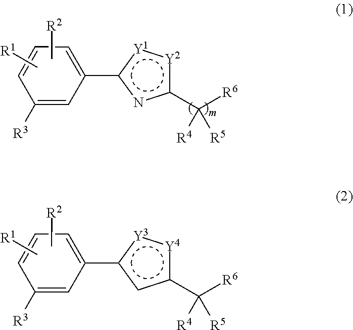

### CROSS-REFERENCE TO RELATED APPLICATIONS

This application claims the benefit of U.S. provisional application Ser. No. 61/168,720, filed Apr. 13, 2009; which is incorporated herein by reference its entirety.

### TECHNICAL FIELD

The present invention relates to compositions and methods for modulating retinol binding to retinol binding protein 4 (RBP4).

### BACKGROUND ART

Vitamin A and its various metabolites play diverse roles in physiology. For example, vitamin A deficiency is the major cause of blindness in children. Excess vitamin-A levels in organs and tissues, such as the eye, may also cause blindness in a variety of retinal diseases, including macular degeneration. Age-related macular degeneration or dystrophy leads to gradual loss of vision, and eventually severe damage to the central vision. Over ten million individuals are estimated to suffer from AMD, and this number is expected to triple over the next decade.

Abnormal levels of vitamin A, and/or its associated transport proteins, retinol binding protein (RBP) and transthyretin (TTR) are also correlated with the manifestation of other diseases, including metabolic disorders. Abnormal levels of retinol were seen in type I and type II diabetic patients, but not in normal patients. Other diseases include idiopathic intracranial hypertension (IIH), and bone-related disorders, including cervical spondylosis, spinal hyperostosis, and diffuse idiopathic skeletal hyperostosis (DISH). In addition, vitamin A and/or its associated transport proteins, particularly TTR, may play a role in protein misfolding and aggregation disease, including Alzheimer's disease and systemic amyloidosis.

To date, there is no effective cure for retinol-related diseases, and there remains a need for methods and compositions to treat these diseases.

### DISCLOSURE OF THE INVENTION

The present invention relates to compositions and methods for modulating retinol binding to retinol binding protein 4 (RBP4).

In one aspect, the present invention provides a compound of Formula (1) or (2):

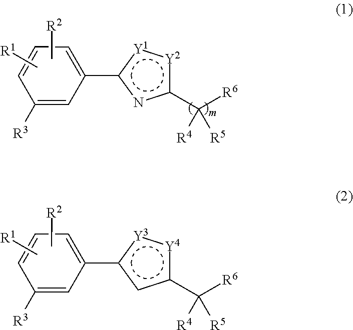

or a physiologically acceptable salt thereof;

wherein R¹ and R² are independently H, halogen, C₁₋₆ alkoxy, or a C₁₋₆ alkyl optionally substituted with halogen, provided R¹ and R² are not both H;

R³ is C₁₋₆ halogenated alkyl;

R⁴ and R⁵ are independently H, OH, C₁₋₆ alkyl, C₁₋₆ alkoxy or C₃₋₇ carbocyclic ring; or R⁴ and R⁵ together may form a 3-6 membered ring;

R⁶ is CO₂R⁷ or a carboxylic acid isostere other than 5,6-dihydro-1,4,2-dioxazinyl;

R⁷ is H or C₁₋₆ alkyl;

one of Y¹ and Y² is S or O and the other is CR⁸ wherein R⁸ is H or C₁₋₆ alkyl; alternatively, one of Y¹ and Y² is N and the other is O;

one of Y³ and Y⁴ is N and the other is O; and

m is 0-1;

provided said compound does not have Formula (1-Q) or (1-R):

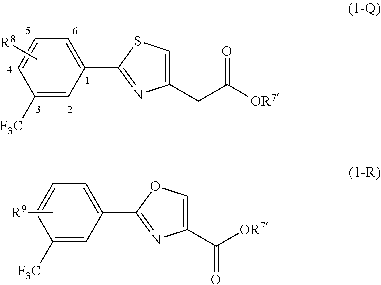

wherein R⁸ is halo at the 6-position of the phenyl ring;

R⁹ is halo; and

each R^{7′} is H or C₁₋₆ alkyl.

In some embodiments, R⁸ in Formula (1Q) is halo at the 2-position of the phenyl ring.

In the above Formula (1) or (2), R¹ may be a substituent at any position of the phenyl ring, and may be selected from halogen, C₁₋₆ alkoxy and C₁₋₆ alkyl optionally substituted with halogen; and R² may be H. In some examples, R⁶ is CO₂R⁷; and R⁷ is H or C₁₋₆ alkyl. In other examples, R⁶ is a carboxylic acid isostere. For example, R⁶ may be a carboxylic acid isostere selected from the group consisting of

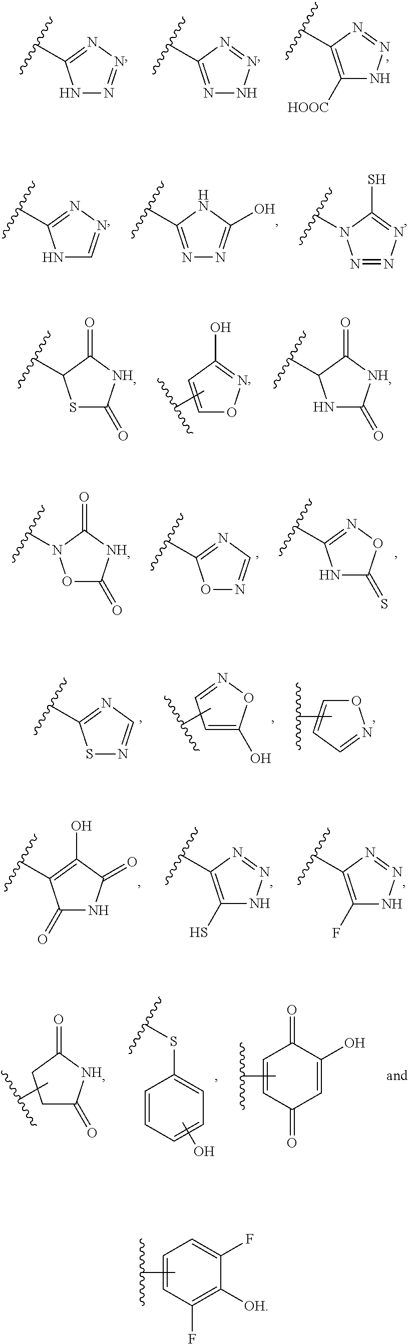

In one embodiment, the invention provides a compound of Formula (1A):

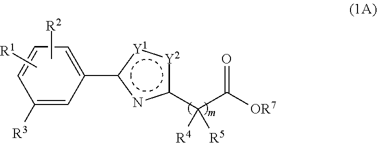

wherein R¹ and R² are halogen; and

R³, R⁴, R⁵, R⁷, Y¹, Y² and m are as defined in Formula (1).

In another embodiment, the invention provides a compound of Formula (1B):

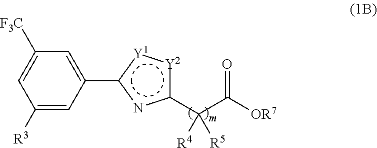

wherein R³, R⁴, R⁵, R⁷, Y¹, Y² and m are as defined in Formula (1).

In any of the above Formula (1), (1A) or (1B), Y¹ may be S or O and Y² is CR⁸, and R⁸ is H or C₁₋₆ alkyl. In other examples, Y² is S or O and Y¹ is CR⁸, and R⁸ is H or C₁₋₆ alkyl. In yet other examples, one of Y¹ is N and the other is O. In yet other examples, m is 1.

In yet another embodiment, the invention provides a compound of Formula (2);

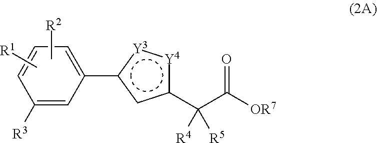

wherein R¹, R², R³, R⁴, R⁵, R⁷, Y³ and Y⁴ are as defined above.

In any of the above Formula (1), (1A), (1B), (2) or (2A), R³ may be CF³. In other examples, R⁴ and R⁵ are H. In other examples, R⁴ is H and R⁵ is OH.

In another aspect, the present invention provides pharmaceutical compositions comprising a compound having Formula (1), (1A), (1B), (2) or (2A), and a physiologically acceptable carrier.

In yet another aspect, the invention provides methods for inhibiting retinol binding to retinol binding protein 4 (RBP4) in a cell, comprising contacting the cell with an effective amount of a compound having Formula (1) or (2),

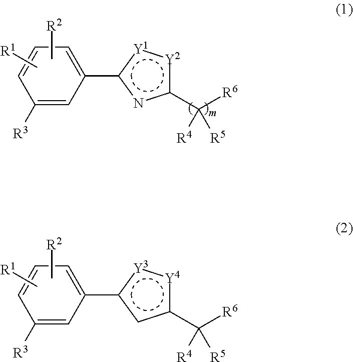

or a physiologically acceptable salt thereof;

wherein R¹ and R² are independently H, halogen, C₁₋₆ alkoxy, or a C₁₋₆ alkyl optionally substituted with halogen, provided R¹ and R² are not both H;

R³ is C₁₋₆ halogenated alkyl;

R⁴ and R⁵ are independently H, OH, C₁₋₆ alkyl, C₁₋₆ alkoxy or C₃₋₇ carbocyclic ring; or R⁴ and R⁵ together may form a 3-6 membered ring;

R⁶ is CO₂R⁷ or a carboxylic acid isostere other than 5,6-dihydro-1,4,2-dioxazinyl;

R⁷ is H or C₁₋₆ alkyl;

one of Y¹ and Y² is S or O and the other is CR⁸ wherein R⁸ is H or C₁₋₆ alkyl; alternatively, one of Y¹ and Y² is N and the other is O;

one of Y³ and Y⁴ is N and the other is O;

m is 0-1;

thereby inhibiting retinol binding to RBP4.

The invention also provides methods for treating a condition mediated by retinol binding to retinol binding protein 4 (RBP4) in a subject suffering therefrom, comprising administering to said subject an effective amount of a compound of Formula (1) or (2),

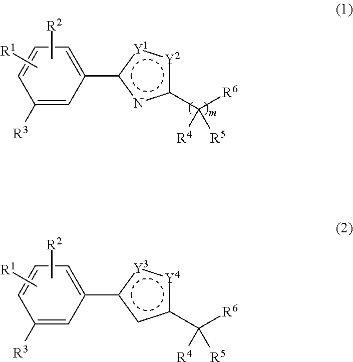

or a physiologically acceptable salt thereof;

wherein R¹ and R² are independently H, halogen, C₁₋₆ alkoxy, or a C₁₋₆ alkyl optionally substituted with halogen, provided R¹ and R² are not both H;

R³ is C₁₋₆ halogenated alkyl;

R⁴ and R⁵ are independently H, OH, C₁₋₆ alkyl, C₁₋₆ alkoxy or C₃₋₇ carbocyclic ring; or R⁴ and R⁵ together may form a 3-6 membered ring;

R⁶ is CO₂R⁷ or a carboxylic acid isostere other than 5,6-dihydro-1,4,2-dioxazinyl;

R⁷ is H or C₁₋₆ alkyl;

one of Y¹ and Y² is S or O and the other is CR⁸ wherein R⁸ is H or C₁₋₆ alkyl; alternatively, one of Y¹ and Y² is N and the other is O;

one of Y³ and Y⁴ is N and the other is O;

m is 0-1;

wherein said condition is macular degeneration or Stargardt's disease.

Furthermore, the invention provides for the use of a compound having Formula (1) or (2):

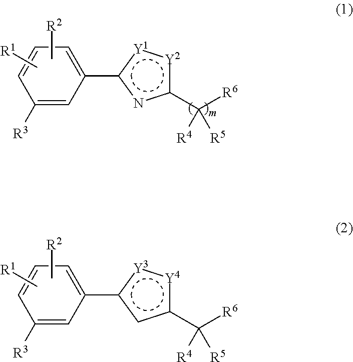

or a physiologically acceptable salt or a pharmaceutical composition thereof, for inhibiting retinol binding to retinol binding protein 4 (RBP4);

wherein R¹ and R² are independently H, halogen, C₁₋₆ alkoxy, or a C₁₋₆ alkyl optionally substituted with halogen, provided R¹ and R² are not both H;

R³ is C₁₋₆ halogenated alkyl;

R⁴ and R⁵ are independently H, OH, C₁₋₆ alkyl, C₁₋₆ alkoxy or C₃₋₇ carbocyclic ring; or R⁴ and R⁵ together may form a 3-6 membered ring;

R⁶ is CO₂R⁷ or a carboxylic acid isostere other than 5,6-dihydro-1,4,2-dioxazinyl;

R⁷ is H or C₁₋₆ alkyl;

one of Y¹ and Y² is S or O and the other is CR⁸ wherein R⁸ is H or C₁₋₆ alkyl; alternatively, one of Y¹ and Y² is N and the other is O;

one of Y³ and Y⁴ is N and the other is O; and

m is 0-1.

The invention also provides for the use of a compound having Formula (1) or (2)

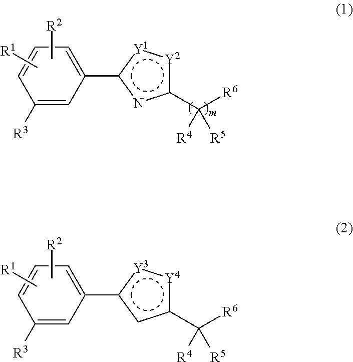

or a physiologically acceptable salt thereof or a pharmaceutical composition thereof, in the manufacture of a medicament for the treatment of macular degeneration or Stargardt's disease;

wherein R¹ and R² are independently H, halogen, C₁₋₆ alkoxy, or a C₁₋₆ alkyl optionally substituted with halogen, provided R¹ and R² are not both H;

R³ is C₁₋₆ halogenated alkyl;

R⁴ and R⁵ are independently H, OH, C₁₋₆ alkyl, C₁₋₆ alkoxy or C₃₋₇ carbocyclic ring; or R⁴ and R⁵ together may form a 3-6 membered ring;

R⁶ is CO₂R⁷ or a carboxylic acid isostere other than 5,6-dihydro-1,4,2-dioxazinyl;

R⁷ is H or C₁₋₆ alkyl;

one of Y¹ and Y² is S or O and the other is CR⁸ wherein R⁸ is H or C₁₋₆ alkyl; alternatively, one of Y¹ and Y² is N and the other is O;

one of Y³ and Y⁴ is N and the other is O; and

m is 0-1.

In the above methods for using the compounds of the invention, the compounds of the invention may be used alone or in combination with a second therapeutic agent, for treating a condition mediated by retinol binding to retinol binding protein 4 (RBP4), wherein said condition is macular degeneration or Stargardt's disease. In some examples, the condition is age-related macular degeneration (AMD), particularly dry or atrophic atrophic AMD.

In the above methods for using the compounds of the invention, a compound having Formula (1), (1A), (1B), (2) or (2A), may be administered to a human or animal subject.

### DEFINITIONS

“Alkyl” refers to a moiety and as a structural element of other groups, for example halo-substituted-alkyl and alkoxy, and may be straight-chained or branched. An optionally substituted alkyl, alkenyl or alkynyl as used herein may be optionally halogenated (e.g., CF₃), or may have one or more carbons that is substituted or replaced with a heteroatom, such as NR, O or S (e.g., —OCH₂CH₂O—, alkylthiols, thioalkoxy, alkylamines, etc).

A “carbocyclic ring” as used herein refers to a saturated or partially unsaturated, monocyclic, fused bicyclic or bridged polycyclic ring containing carbon atoms, which may optionally be substituted, for example, with ═O. Examples of carbocyclic rings include but are not limited to cyclopropyl, cyclobutyl, cyclopentyl, cyclohexyl, cyclopropylene, cyclohexanone, etc.

A “heterocyclic ring” as used herein is as defined for a carbocyclic ring above, wherein one or more ring carbons is a heteroatom. For example, a heterocyclic ring may contain N, O, S, —N═, —S—, —S(O), —S(O)₂—, or —NR— wherein R may be hydrogen, C₁₋₄alkyl or a protecting group. Examples of heterocyclic rings include but are not limited to morpholino, pyrrolidinyl, pyrrolidinyl-2-one, piperazinyl, piperidinyl, piperidinylone, 1,4-dioxa-8-aza-spiro[4.5]dec-8-yl, etc.

As used herein, an H atom in any substituent groups (e.g., CH₂) encompasses all suitable isotopic variations, e.g., H, ²H and ³H.

“Isosteres” are different compounds that have different molecular formula but exhibit the same or similar properties. The term “carboxylic acid isostere” refers to compounds that mimic the properties of a carboxylic acid even though they have a different molecular formula. Examples of suitable carboxylic acid isosteres include but are not limited to 5-7 membered carbocycles or heterocycles containing any combination of CH₂, O S, or N in any chemically stable oxidation state, where any of the atoms of said ring structure are optionally substituted in one or more positions. Particular carboxylic acid isosteres for use in the compounds of the invention include but are not limited to

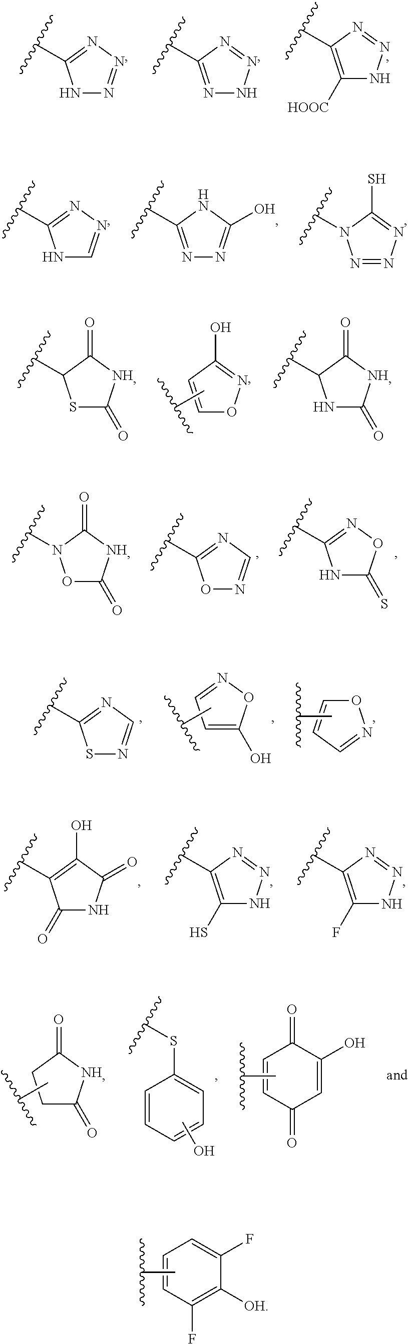

Other carboxylic acid isosteres contemplated by the present invention include—SO₃H, —SO₂HNR⁸, —PO₂ (R⁸)₂, —CN, —PO₃(R⁸)₂, —OR⁸, —SR⁸, —NHCOR⁸, —N(R⁸)₂, —CON(R⁸)₂, —CONH(O)R⁸, —CONHNHSO₂R⁸, —COHNSO₂R⁸, and —CONR⁸CN, wherein R⁸ is H, C₁₋₆ alkyl, aryl, heteroaryl, carbocycle or heterocycle.

The terms “co-administration” or “combined administration” or the like as used herein are meant to encompass administration of the selected therapeutic agents to a single subject (e.g., a patient), and are intended to include treatment regimens in which the agents are not necessarily administered by the same route of administration or at the same time.

The term “pharmaceutical combination” as used herein refers to a product obtained from mixing or combining active ingredients, and includes both fixed and non-fixed combinations of the active ingredients. The term “fixed combination” means that the active ingredients, e.g. a compound of Formula (1) and a co-agent, are both administered to a patient simultaneously in the form of a single entity or dosage. The term “non-fixed combination” means that the active ingredients, e.g. a compound of Formula (1) and a co-agent, are both administered to a patient as separate entities either simultaneously, concurrently or sequentially with no specific time limits, wherein such administration provides therapeutically effective levels of the active ingredients in the body of the patient. The latter also applies to cocktail therapy, e.g. the administration of three or more active ingredients.

The term “therapeutically effective amount” means the amount of the subject compound that will elicit a biological or medical response in a cell, tissue, organ, system, animal or human that is being sought by the researcher, veterinarian, medical doctor or other clinician.

The term “administration” or “administering” of the subject compound means providing a compound of the invention and prodrugs thereof to a subject in need of treatment.

As used herein, the term “age-related macular degeneration or dystrophy” (ARMD) encompasses wet and dry forms of ARMD. The dry form of ARMD is also known as atrophic, nonexudative, or drusenoid (age-related) macular degeneration. The wet form of ARMD is also known as exudative or neovascular (age-related) macular degeneration. The macular dystrophies include Stargardt Disease, also known as Stargardt Macular Dystrophy or Fundus Flavimaculatus, which is the most frequently encountered juvenile onset form of macular dystrophy.

### MODES OF CARRYING OUT THE INVENTION

The present invention relates to compositions and methods for treating retinol-related disease by modulating retinol binding to retinol binding protein.

In one aspect, the present invention provides a compound of Formula (1) or (2):

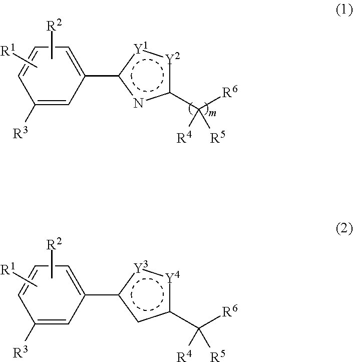

or a physiologically acceptable salt thereof;

wherein R¹ and R² are independently H, halogen, C₁₋₆ alkoxy, or a C₁₋₆ alkyl optionally substituted with halogen, provided R¹ and R² are not both H;

R³ is C₁₋₆ halogenated alkyl;

R⁴ and R⁵ are independently H, OH, C₁₋₆ alkyl, C₁₋₆ alkoxy or C₃₋₇ carbocyclic ring; or R⁴ and R⁵ together may form a 3-6 membered ring;

R⁶ is CO₂R⁷ or a carboxylic acid isostere other than 5,6-dihydro-1,4,2-dioxazinyl;

R⁷ is H or C₁₋₆ alkyl;

one of Y¹ and Y² is S or O and the other is CR⁸ wherein R⁸ is H or C₁₋₆ alkyl; alternatively, one of Y¹ and Y² is N and the other is O;

one of Y³ and Y⁴ is N and the other is O;

m is 0-1;

provided said compound does not have Formula (1-Q) or (1-R):

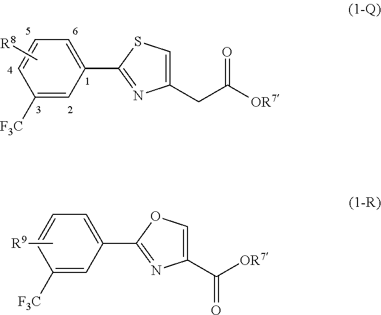

wherein R⁸ is halo at the 6-position of the phenyl ring;

R⁹ is halo; and

each R^{7′} is H or C₁₋₆ alkyl.

In one embodiment, the invention provides a compound of Formula (1A):

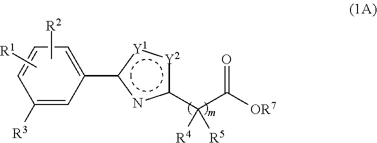

wherein R¹ and R² are halogen; and

R³, R⁴, R⁵, R⁷, Y¹, Y² and m are as defined in Formula (1).

In another embodiment, the invention provides a compound of Formula (1B):

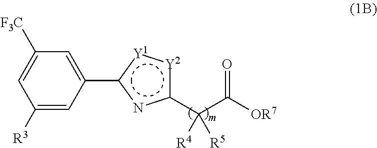

wherein R³, R⁴, R⁵, R⁷, Y¹, Y² and m are as defined in Formula (1).

In yet another embodiment, the invention provides a compound of Formula (2A);

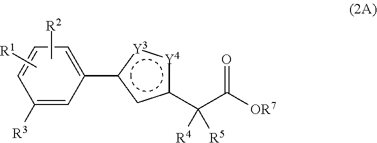

wherein R¹, R², R³, R⁴, R⁵, R⁷, Y³ and Y⁴ are as defined above.

In each of the above formula, any asymmetric carbon atoms may be present in the (R)-, (S)- or (R,S)-configuration. The compounds may thus be present as mixtures of isomers or as pure isomers, for example, as pure enantiomers or diastereomers. The invention further encompasses possible tautomers of the inventive compounds.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F³¹P, ³²P, ³⁵S, ³⁶Cl, ¹²⁵I respectively.

The invention includes various isotopically labeled compounds as defined herein, for example, those into which radioactive isotopes such as ³H, ¹³C, and ¹⁴C, are present. Such isotopically labelled compounds are useful in metabolic studies (with, for example, ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In other examples, an ¹⁸F or labeled compound may be used for PET or SPECT studies. Isotopic variations of the compounds have the potential to change a compound's metabolic fate and/or create small changes in physical properties such as hydrophobicity, and the like. Isotopic variations also have the potential to enhance efficacy and safety, enhance bioavailability and half-life, alter protein binding, change biodistribution, increase the proportion of active metabolites and/or decrease the formation of reactive or toxic metabolites. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Pharmacology and Utility

The present invention provides compositions and methods for modulating retinol binding to retinol binding protein 4 (RBP4). RBP4 is a circulatory protein that is part of an extracellular transport system for retinol. RBP4 is synthesized in an apo form in the rough endoplasmic reticulum, but is not efficiently transferred out of the endoplasmic reticulum until it is complexed with retinol. Furthermore, RBP4 is predominately found in the serum bound to transthyretin (TTR). TTR itself can bind two molecules of thyroid protein, but in the context of retinal homeostasis, is thought to prevent RBP4 from being excreted during plasma filtration in the kidney. Therefore, the activity level of RBP4 can be altered by changing the level of RBP4 produced or maintained in the body, which in turn can be altered by changing 1) the rate of production of nascent RBP4, 2) the ability of RBP4 to interact with retinol, 3) the ability of RBP4 to interact with TTR and 4) the half life of RBP4 in the body. In addition, RBP4 activity can be altered by changing the ability of RBP4 to deliver retinol to the cells such that, for example, retinal dependent signaling is affected.

The present invention also provides compositions and methods for the treatment of a condition mediated by retinol binding to retinol binding protein 4 (RBP4). In particular embodiments, the present invention provides compositions and methods for the treatment of macular degeneration and dystrophies. It is also contemplated that the compositions of the present invention may be used for the treatment of a condition mediated by retinol binding to retinol binding protein (RBP), including metabolic disorders associated with abnormal retinol levels and other retinol-related diseases.

Macular Degeneration and Dystrophies

Macular degeneration (also referred to as retinal degeneration) is a disease of the eye that involves deterioration of the macula, the central portion of the retina. Approximately 85% to 90% of the cases of macular degeneration are the “dry” (atrophic or non-neovascular) type. In dry macular degeneration, the deterioration of the retina is associated with the formation of small yellow deposits (i.e., drusen), under the macula; in addition, the accumulation of lipofuscin in the RPE leads to geographic atrophy. This phenomena leads to a thinning and drying out of the macula. The location and amount of thinning in the retina caused by the drusen directly correlates to the amount of central vision loss. Degeneration of the pigmented layer of the retina and photoreceptors overlying drusen become atrophic and can cause a slow loss of central vision.

In “wet” macular degeneration, new blood vessels form (i.e., neovascularization) to improve the blood supply to retinal tissue beneath the macula, a portion of the retina that is responsible for our sharp central vision. The new vessels are easily damaged and sometimes rupture, causing bleeding and injury to the surrounding tissue. Neovascularization can lead to rapid loss of vision and eventual scarring of the retinal tissues. This scar tissue and blood produces a dark, distorted area in the vision, often rendering the eye legally blind. Although wet macular degeneration only occurs in about 10 percent of all macular degeneration cases, it accounts for approximately 90% of macular degeneration-related blindness.

Wet macular degeneration usually starts with distortion in the central field of vision. Straight lines become wavy. Many people with macular degeneration also report having blurred vision and blank spots in their visual field. Growth promoting proteins called vascular endothelial growth factor, or VEGF, have been targeted for triggering this abnormal vessel growth in the eye. This discovery has lead to aggressive research of experimental drugs that inhibit or block VEGF. Studies have shown that anti-VEGF agents can be used to block and prevent abnormal blood vessel growth. Such anti-VEGF agents stop or inhibit VEGF stimulation, so there is less growth of blood vessels. Such anti-VEGF agents may also be successful in anti-angiogenesis or blocking VEGF's ability to induce blood vessel growth beneath the retina, as well as blood vessel leakiness.

In addition, several types of macular degenerations affect children, teenagers or adults, and are commonly known as early onset or juvenile macular degeneration. Many of these types are hereditary and are looked upon as macular dystrophies instead of degeneration. Some examples of macular dystrophies include: Cone-Rod Dystrophy, Corneal Dystrophy, Fuch's Dystrophy, Sorsby's Macular Dystrophy, Best Disease, and Juvenile Retinoschisis, as well as Stargardt Disease.

Stargardt Disease

Stargardt Disease is a macular dystrophy that manifests as a recessive form of macular degeneration with an onset during childhood. See e.g., Allikmets et al., Science, 277:1805-07 (1997). Stargardt Disease is characterized clinically by progressive loss of central vision and progressive atrophy of the RPE overlying the macula. Mutations in the human ABCA4 gene for Rim Protein (RmP) are responsible for Stargardt Disease. Early in the disease course, patients show delayed dark adaptation but otherwise normal rod function. Histologically, Stargardt Disease is associated with deposition of lipofuscin pigment granules in RPE cells.

Besides Stargardt Disease, mutations in ABCA4 have been implicated in recessive retinitis pigmentosa, recessive cone-rod dystrophy, and non-exudative age-related macular degeneration (AMD), see e.g., Lewis et al., Am. J. Hum. Genet., 64:422-34 (1999), although the prevalence of ABCA4 mutations in AMD is still uncertain. See Allikmets, Am. J. Hum. Gen., 67:793-799 (2000) Similar to Stargardt Disease, these diseases are associated with delayed rod dark-adaptation. Lipofuscin deposition in RPE cells is also seen prominently in AMD, see Kliffen et al., Microsc. Res. Tech., 36:106-22 (1997), and in some cases of retinitis pigmentosa and cone-rod dystrophy.

Travis et al. (Annu. Rev. Pharmocol. Toxicol. 2007. 47:8.1-8.44) present the relationship between the disruption of the formation of the RBP4-TTR-vitamin A complex (inhibiting the transport of vitamin A from serum to the eye via this complex) and the subsequent reduction of A2E levels with ophthalmic diseases, including AMD and Stargardt's Disease. Although the mechanism is not required to practice the invention, disruption of the RBP4-TTR complex results in decreased plasma retinol levels, decreased delivery of retinol to the eye and decreased formation of A2E. In certain embodiments, compounds of Formula 1 and Formula 2 provided herein decrease the level of A2E. Thus, treatment of AMD or Stargardt's Disease patients with such compounds that disrupt the RBP4-TTR complex and lower plasma RBP4 levels should reduce the formation of A2E and prevent further loss of vision. Simple displacement of retinol from RBP4 may also be effective for reducing retinol delivery to the eye and reduces A2E production.

Administration and Pharmaceutical Compositions

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention may be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form.

Pharmaceutical compositions comprising a compound of the present invention in free form or in a pharmaceutically acceptable salt form in association with at least one pharmaceutically acceptable carrier or diluent may be manufactured in a conventional manner by mixing, granulating or coating methods. For example, oral compositions may be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets, together with c) binders, e.g., magnesium aluminum silicate, starch paste, gelatin, tragacanth, methylcellulose, sodium carboxymethylcellulose and or polyvinylpyrrolidone; and if desired, d) disintegrants, e.g., starches, agar, alginic acid or its sodium salt, or effervescent mixtures; and/or e) absorbents, colorants, flavors and sweeteners. Injectable compositions may be aqueous isotonic solutions or suspensions, and suppositories may be prepared from fatty emulsions or suspensions.

The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier may include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, may be aqueous solutions, ointments, creams or gels well-known in the art. Such may contain solubilizers, stabilizers, tonicity enhancing agents, buffers and preservatives.

Compounds of the invention may be administered in therapeutically effective amounts in combination with one or more therapeutic agents (pharmaceutical combinations). For example, synergistic effects may occur with other immunomodulatory or anti-inflammatory substances, for example when used in combination with cyclosporin, rapamycin, or ascomycin, or immunosuppressant analogues thereof, for example cyclosporin A (CsA), cyclosporin G, FK-506, rapamycin, or comparable compounds, corticosteroids, cyclophosphamide, azathioprine, methotrexate, brequinar, leflunomide, mizoribine, mycophenolic acid, mycophenolate mofetil, 15-deoxyspergualin, immunosuppressant antibodies, especially monoclonal antibodies for leukocyte receptors, for example MHC, CD2, CD3, CD4, CD7, CD25, CD28, B7, CD45, CD58 or their ligands, or other immunomodulatory compounds, such as CTLA41g. Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit may comprise instructions for its administration.

Processes for Making Compounds of the Invention

In general, compounds having Formula (1) may be prepared following any one of the synthetic methodologies described in Scheme 1-9, infra. In the reactions described, reactive functional groups, for example hydroxy, amino, imino, thio or carboxy groups, where these are desired in the final product, may be protected to avoid their unwanted participation in the reactions. Conventional protecting groups may be used in accordance with standard practice (see e.g., T. W. Greene and P. G. M. Wuts in “Protective Groups in Organic Chemistry”, John Wiley and Sons, 1991). Suitable leaving groups for use in the synthetic methodologies described include halogen leaving groups (e.g., chloro or bromo), and other conventional leaving groups within the knowledge of those skilled in the art.

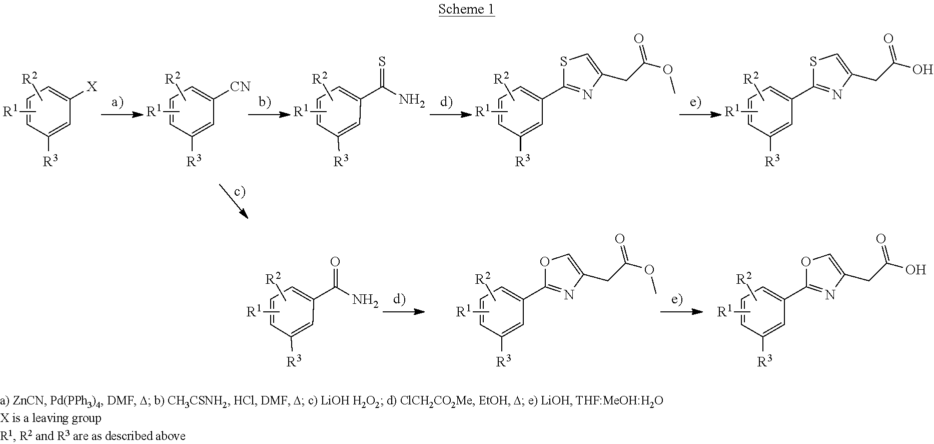

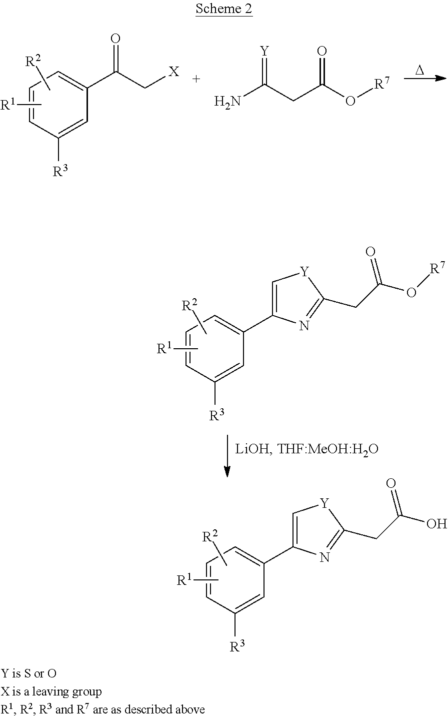

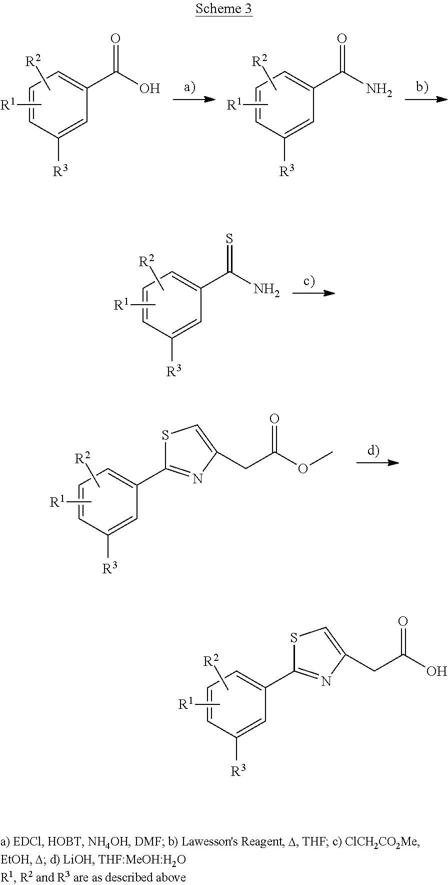

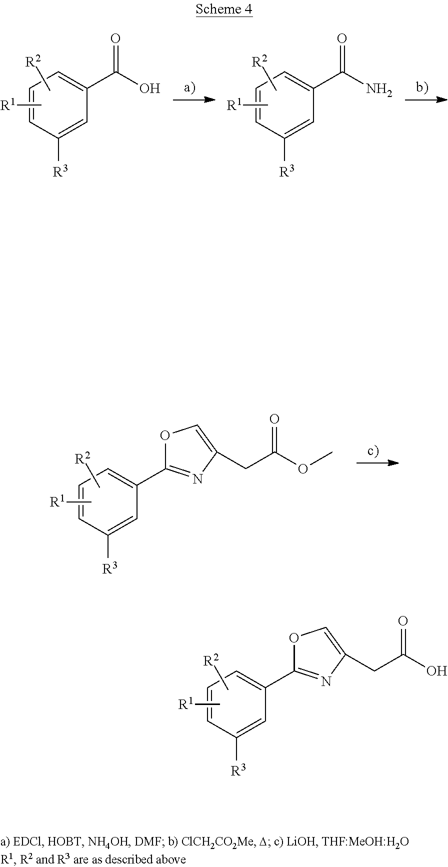

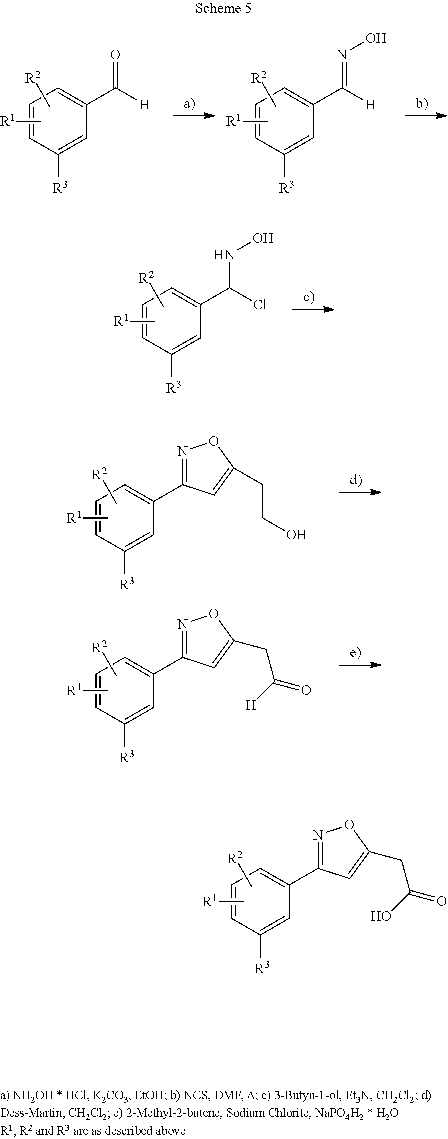

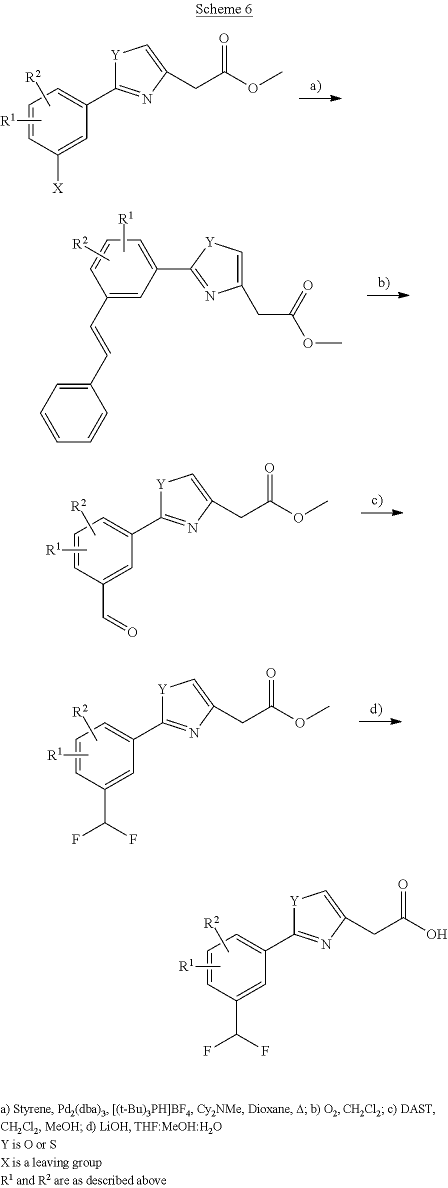

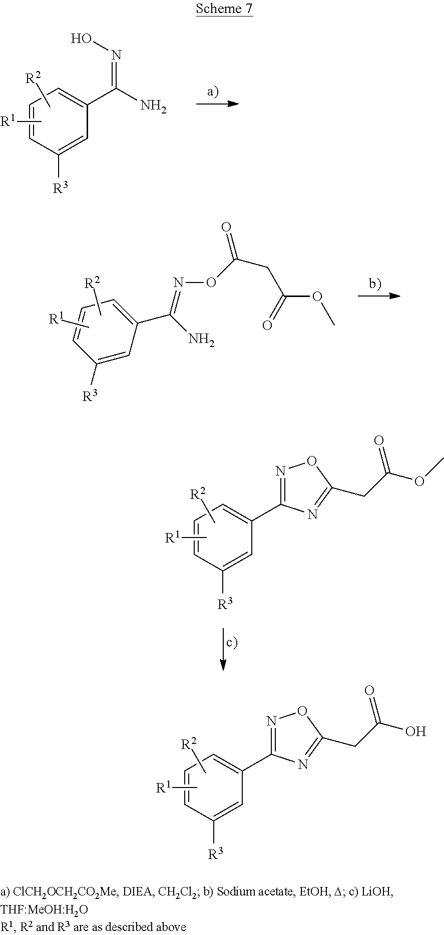

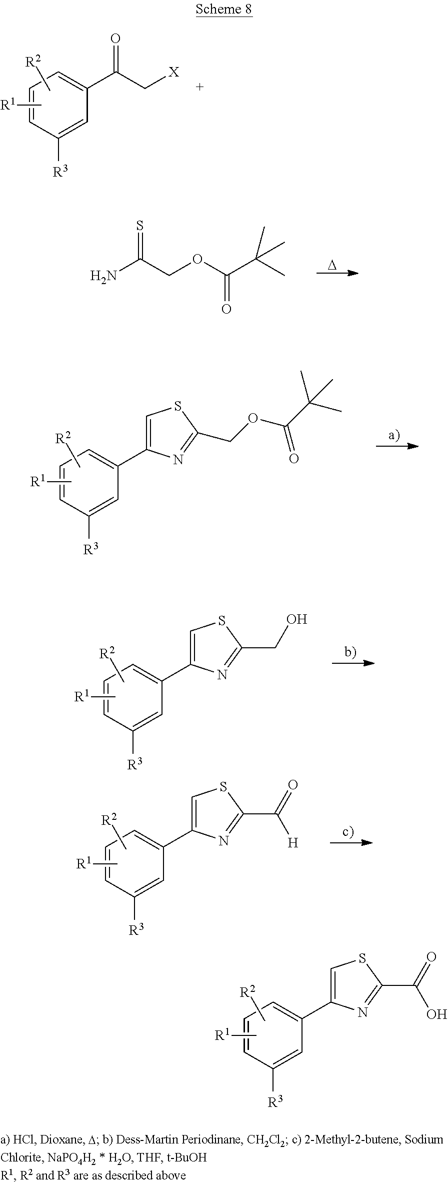

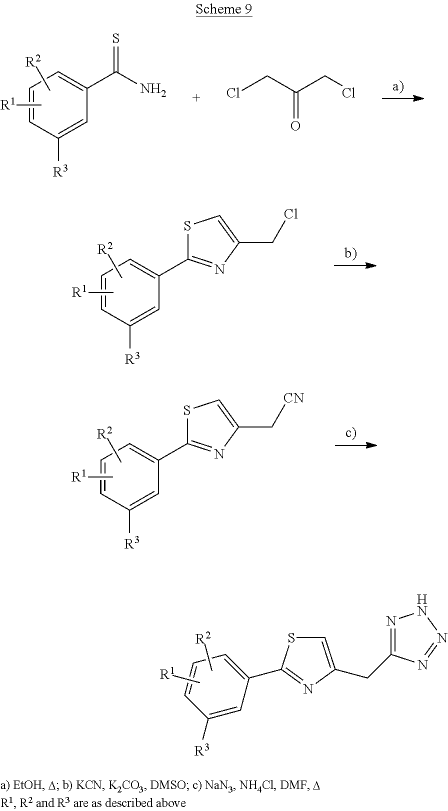

The compounds of the invention, including their salts, are also obtainable in the form of hydrates, or their crystals may include for example the solvent used for crystallization (present as solvates). Salts can usually be converted to compounds in free form, e.g., by treating with suitable basic agents, for example with alkali metal carbonates, alkali metal hydrogen carbonates, or alkali metal hydroxides, such as potassium carbonate or sodium hydroxide. A compound of the invention in a base addition salt form may be converted to the corresponding free acid by treating with a suitable acid (e.g., hydrochloric acid, etc.). In view of the close relationship between the novel compounds in free form and those in the form of their salts, including those salts that may be used as intermediates, for example in the purification or identification of the novel compounds, any reference to the free compounds is to be understood as referring also to the corresponding salts, as appropriate.

Salts of the inventive compounds with a salt-forming group may be prepared in a manner known per se. Acid addition salts of compounds of Formula (1), (1A), (1B), (2) or (2A) may thus be obtained by treatment with an acid or with a suitable anion exchange reagent. Pharmaceutically acceptable salts of the compounds of the invention may be formed, for example, as acid addition salts, with organic or inorganic acids, from compounds of Formula (1), (1A), (1B), (2) or (2A) with a basic nitrogen atom.

Suitable inorganic acids include, but are not limited to, halogen acids, such as hydrochloric acid, sulfuric acid, or phosphoric acid. Suitable organic acids include, but are not limited to, carboxylic, phosphoric, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid,-malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4 aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4 methylbenzenesulfonic acid, methylsulfuric acid, ethylsulfuric acid, dodecylsulfuric acid, N cyclohexylsulfamic acid, N-methyl-, N-ethyl- or N-propyl-sulfamic acid, or other organic protonic acids, such as ascorbic acid. For isolation or purification purposes, it is also possible to use pharmaceutically unacceptable salts, for example picrates or perchlorates. For therapeutic use, only pharmaceutically acceptable salts or free compounds are employed (where applicable in the form of pharmaceutical preparations).

Compounds of the invention in unoxidized form may be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention may be prepared by methods known to those of ordinary skill in the art (e.g., for further details see Saulnier et al., (1994), Bioorganic and Medicinal Chemistry Letters, Vol. 4, p. 1985). For example, appropriate prodrugs may be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention may be made by means known to those of ordinary skill in the art. A detailed description of techniques applicable to the creation of protecting groups and their removal may be found in T. W. Greene, “Protecting Groups in Organic Chemistry”, 3rd edition, John Wiley and Sons, Inc., 1999.

Compounds of the invention may be prepared as their individual stereoisomers by reacting a racemic mixture of the compound with an optically active resolving agent to form a pair of diastereoisomeric compounds, separating the diastereomers and recovering the optically pure enantiomers. Resolution of enantiomers may be carried out using covalent diastereomeric derivatives of the compounds of the invention, or by using dissociable complexes (e.g., crystalline diastereomeric salts). Diastereomers have distinct physical properties (e.g., melting points, boiling points, solubilities, reactivity, etc.) and may be readily separated by taking advantage of these dissimilarities. The diastereomers may be separated by fractionated crystallization, chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture may be found in Jean Jacques, Andre Collet, Samuel H. Wilen, “Enantiomers, Racemates and Resolutions”, John Wiley And Sons, Inc., 1981.

In summary, the compounds of the invention may be made by a process as described in the Examples; and

(a) optionally converting a compound of the invention into a pharmaceutically acceptable salt;

(b) optionally converting a salt form of a compound of the invention to a non-salt form;

(c) optionally converting an unoxidized form of a compound of the invention into a pharmaceutically acceptable N-oxide;

(d) optionally converting an N-oxide form of a compound of the invention to its unoxidized form;

(e) optionally resolving an individual isomer of a compound of the invention from a mixture of isomers;

(f) optionally converting a non-derivatized compound of the invention into a pharmaceutically acceptable prodrug derivative; and

(g) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter. One of skill in the art will appreciate that the above transformations are only representative of methods for preparation of the compounds of the present invention, and that other well known methods can similarly be used. The present invention is further exemplified, but not limited, by the following and Examples that illustrate the preparation of the compounds of the invention.

### Example 1

### Methyl 2-(2-(3,5-bis(trifluoromethyl)phenyl)thiazol-4-yl)acetate

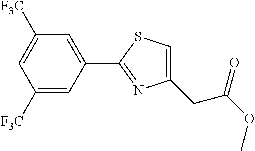

To a sealed vial was added 3,5-bis(trifluoromethyl)benzothioamide (60 mg, 0.22 mmol), methyl-4-chloroacetoacetate (33 mg, 0.22 mmol) and ethanol (1 mL). After stiffing at 180° C. in the microwave for 10 minutes, the volatile reagents were removed under reduced pressure. The compound was purified via silica gel chromatography (0%-35% ethyl acetate/hexanes) to give the desired product.

### Example 2

### 2-(2-(3,5-Bis(trifluoromethyl)phenyl)thiazol-4-yl)acetic acid

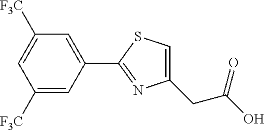

To a vial was added methyl 2-(2-(3,5-bis(trifluoromethyl)phenyl)thiazol-4-yl)acetate (78 mg, 0.22 mmol), LiOH.H₂O (26 mg, 0.66 mmol) and THF:MeOH:H₂O (10:1:10, 1 mL). The reaction was stirred at ambient temperature for 4 h and then quenched with 10% Citric Acid and extracted with EtOAc (3×). Organic layers were combined and washed with H₂O, brine and dried over MgSO₄. The volatile organic solvents were removed under reduced pressure. The product was recrystallized from EtOH/H₂O.

### Example 3

### Methyl 2-(3-(3,5-bis(trifluoromethyl)phenyl)-1,2,4-oxadiazol-5-yl)acetate

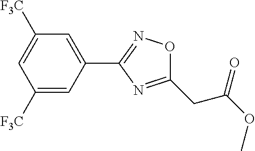

To a vial was added 3,5-bis(trifluoromethyl)benzamidoxime (500 mg, 1.84 mmol), DIPEA (641 μL, 3.86 mmol) and CH₂Cl₂ (15 mL), and the mixture was cooled to 0° C. in an ice/water bath. After 10 min of immersion, methyl malonyl chloride (236 μL, 2.21 mmol) was added, and the reaction was removed from the cooling bath and stirred at ambient temperature overnight. The reaction was diluted with water (10 mL) and then a standard aqueous acidic workup using EtOAc, 10% Citric acid, and brine was employed. The volatile organic solvents were removed under reduced pressure. The reaction mixture was dissolved in dioxane (3 mL), and catalytic TBAF was added to the reaction mixture in a microwave vessel. The reaction mixture was heated to 150° C. for 10 min Upon cooling to ambient temperature, the volatile organic solvents were removed under reduced pressure. The organic layer residue was dissolved in DMSO, and the product purified from the reaction mixture via preparative HPLC.

### Example 4

### Methyl 2-(2-(3,5-bis(trifluoromethyl)phenyl)oxazol-4-yl)acetate

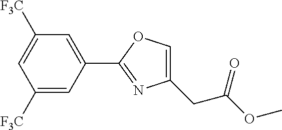

To a sealed vial under argon was added 3,5-bis-trifluoromethyl-benzamide (70 mg, 0.27 mmol) and methyl-4-chloroacetoacetate (123 mg, 0.82 mmol). After stirring neat at 120° C. for 2 hours, the reaction was cooled to room temperature and diluted with H₂O. The aqueous layer was then extracted with EtOA_{C} (3×). The organic extracts were then combined, washed with brine, dried over MgSO₄, and concentrated under reduced pressure. The compound was purified via silica gel chromatography (0%-35% ethyl acetate/hexanes) to give the desired product.

### Example 5

### Methyl 2-(4-(3,5-bis(trifluoromethyl)phenyl)oxazol-2-yl)acetate

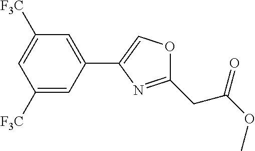

To a sealed vial under argon was added 1-(3,5-bis-trifluoromethyl-phenyl)-2-bromo-ethanone (50 mg, 0.15 mmol) and methyl malonate monoamide (52 mg, 0.45 mmol). After stirring neat at 120° C. for 2 hours, the reaction was cooled to room temperature and diluted with H₂O. The aqueous layer was then extracted with EtOAC (3×). The organic extracts were then combined, washed with brine, dried over MgSO₄, and concentrated under reduced pressure. The compound was purified via silica gel chromatography in a 0%-35% EtOAc/hexanes gradient to give the desired product.

### Example 6

### 2-(2-(3,5-Bis(trifluoromethyl)phenyl)oxazol-4-yl) acetic acid

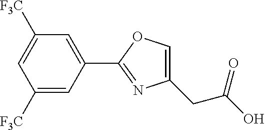

Example 6 was prepared from methyl 2-(2-(3,5-bis(trifluoromethyl)phenyl)oxazol-4-yl)acetate (example 4) according to the method described in example 2. The product was recrystallized from EtOH/H₂O.

### Example 7

### 2-(4-(3,5-Bis(trifluoromethyl)phenyl)oxazol-2-yl)acetic acid

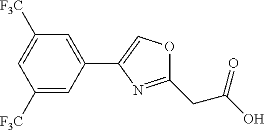

Example 7 was prepared from methyl 2-(4-(3,5-bis(trifluoromethyl)phenyl)oxazol-2-yl)acetate (example 5) according to method described in example 2. The organic layer residue was dissolved in DMSO, and the product purified from the reaction mixture via preparative HPLC.

### Example 8

### 2-(2-(3-Chloro-5-(trifluoromethyl)phenyl)thiazol-4-yl)acetic acid

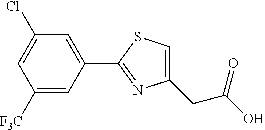

Example 8 was prepared according to scheme 1 starting with 3-chloro-5-trifluoromethyl-benzonitrile. To a 100 mL flask is added 3-chloro-5-trifluoromethyl-benzonitrile (2 g, 9.7 mmol), thioacetamide (1.9 g, 3.9 mmol), 4N HCl (9.2 mL, 36.9 mL) and DMF (20 mL). The reaction was heated at 95° C. overnight. Upon cooling, the reaction mixture was diluted with water and sat NaHCO₃. A standard DMF organic workup gave 3-chloro-5-(trifluoromethyl)benzothioamide as a yellowish solid after removal of all volatiles. Compound was purified via silica gel chromatography (5%-45% EtOAc/Hexanes gradient). The desired thioamide (100 mg, 0.42 mmol) was reacted with methyl-4-chloroacetoacetate (63 mg, 0.42 mmol) as described in the synthesis of example 1 to afford the desired thiazole. The methyl ester was saponified as described in the synthesis of example 2. The product was recrystallized from EtOH/H₂O.

### Example 9

### 2-(4-(3,5-Bis(trifluoromethyl)phenyl)-5-methylthiazol-2-yl)acetic acid

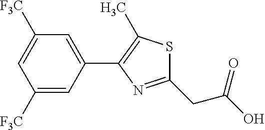

To a sealed vial was added 1-(3,5-bis-trifluoromethyl-phenyl)-2-bromo-propan-1-one (70 mg, 0.20 mmol), ethyl-3-amino-2-thioxyproponoate (29 mg, 0.20 mmol) and ethanol (1 mL). After stirring at 180° C. in the microwave for 10 minutes, the volatile reagents were removed under reduced pressure. The compound was purified via silica gel chromatography (0%-35% EtOAc/hexanes) to give the desired product. The ethyl ester was saponified as described in the synthesis of example 2. The organic layer residue was dissolved in DMSO, and the product purified from the reaction mixture via preparative HPLC.

### Example 10

### 2-(2-(4-Chloro-3-(trifluoromethyl)phenyl)thiazol-4-yl)acetic acid

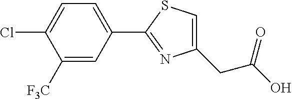

Example 10 was prepared with 4-chloro-3-(trifluoromethyl)benzonitrile according to the synthesis described in example 8. The methyl ester was saponified as described in the synthesis of example 2. The product was recrystallized from EtOH/H₂O.

### Example 11

### 2-(4-(3,5-Bis(trifluoromethyl)phenyl)-5-methyloxazol-2-yl)acetic acid

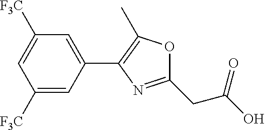

Example 11 was prepared from 1-(3,5-bis-trifluoromethyl-phenyl)-2-bromo-propan-1-one (70 mg, 0.20 mmol) and methyl malonate monoamide (70 mg, 0.6 mmol) according to the synthesis described in example 5. The methyl ester was saponified as described in the synthesis of example 2. The organic layer residue was dissolved in DMSO, and the product purified from the reaction mixture via preparative HPLC.

### Example 12

### 2-(4-(3-Fluoro-5-(trifluoromethyl)phenyl)oxazol-2-yl)acetic acid

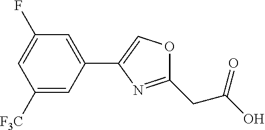

Example 12 was prepared from 2-bromo-1-(3-fluoro-5-trifluoromethyl-phenyl)-ethanone (200 mg, 0.70 mmol) and methyl malonate monoamide (246 mg, 2.1 mmol) according to the synthesis described in example 5. The methyl ester was saponified as described in the synthesis of example 2. The organic layer residue was dissolved in DMSO, and the product purified from the reaction mixture via preparative HPLC.

### Example 13

### 2-(4-(3-Fluoro-5-(trifluoromethyl)phenyl)thiazol-2-yl)acetic acid

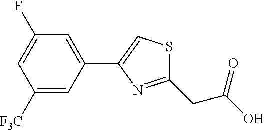

Example 13 was prepared from 1-(3,5-bis-trifluoromethyl-phenyl)-2-bromo-propan-1-one (70 mg, 0.20 mmol) and ethyl-3-amino-2-thioxyproponoate (29 mg, 0.20 mmol) according to the synthesis described in example 9. The ethyl ester was saponified as described in the synthesis of example 2. The product was recrystallized from EtOH/H₂O.

### Example 14

### 2-(2-(3-Methoxy-5-(trifluoromethyl)phenyl)thiazol-4-yl)acetic acid

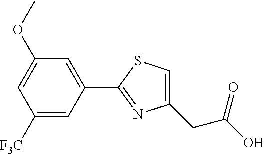

Example 14 was prepared from 3-methoxy-5-trifluoromethyl-thiobenzamide (70 mg, 0.30 mmol) and methyl methyl-4-chloroacetoacetate (45 mg, 0.3 mmol) according to the synthesis described in example 1. The methyl ester was saponified as described in the synthesis of example 2. The product was recrystallized from EtOH/H₂O.

### Example 15

### 2-(2-(3-Methoxy-5-(trifluoromethyl)phenyl)oxazol-4-yl)acetic acid

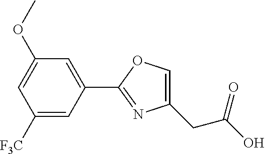

To a sealed vial was added 3-methoxy-5-trifluoromethyl-benzoic acid (1.1 g, 5.0 mmol), HOBt (810 mg, 6.0 mmol), EDCI (1.0 g, 5.5 mmol), and DMF (12 mL). The reaction was stirred under argon at ambient temperature for 1 h, and then NH₄OH (aq) (3 mL) was added in one portion. The reaction was allowed to stir overnight at ambient temperature. Upon completion, the reaction was quenched with 10% citric acid and extracted with EtOAc. Organic layers were combined and washed with H₂O and brine, dried over MgSO₄ and concentrated to give the desired 3-methoxy-5-trifluoromethyl-benzamide, which was then used crude in the next step. The amide (150 mg, 0.68 mmol) was reacted with methyl-4-chloroacetoacetate (86 mg, 0.57 mmol) according to the synthesis in example 4. The methyl ester was saponified as described in the synthesis of example 2. The product was recrystallized from EtOH/H₂O.

### Example 16

### 2-(2-(3,5-Bis(trifluoromethyl)phenyl)thiazol-4-yl)-2-hydroxyacetic acid

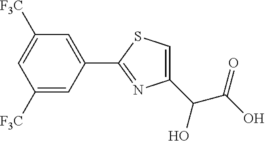

To a sealed vial was added 3,5-bis(trifluoromethyl)benzothioamide (150 mg, 0.55 mmol), 1-acetoxy-3-chloroacetone (83 mg, 0.55 mmol) and ethanol (1 mL). After stirring at 180° C. in the microwave for 10 minutes, the volatile reagents were removed under reduced pressure. The compound was purified via silica gel chromatography (5%-45% EtOAc/hexanes) to give a mixture of the desired acetal and alcohol. The acetal was saponified as described in the synthesis of example 2, and the resulting alcohol was combined with the alcohol from the previous step.

To a sealed vial was added the alcohol (50 mg, 0.15 mmol), Dess-Martin Periodinane (76 mg, 0.18 mmol) and CH₂Cl₂ (1 mL). The reaction was stirred for 2 hr at ambient temperature. Upon completion, the reaction was quenched with sat Na₂S₂O₃ and sat NaHCO₃ and allowed to stir for an additional 10 min The CH₂Cl₂ was then removed under reduced pressure and the aqueous layer extracted with EtOAc. Organics were combined and washed with H₂O and brine, then dried over MgSO₄ and concentrated under reduced pressure to give the desired aldehyde, which was used crude in the next step. A solution of KCN in H₂O was added to a solution of the aldehyde in THF in one portion with stirring. The mixture was stirred at 50° C. for 3 hr, then THF removed under reduced pressure. To the resulting slurry was added concentrated HCl (1 mL) and the reaction stirred for 30 min Upon completion, the mixture was extracted with EtOAc (3×). Organic layers were then combined and washed with H₂O, Brine, dried over MgSO₄ and concentrated under reduced pressure. The organic layer residue was dissolved in DMSO, and the product purified from the reaction mixture via preparative HPLC.

### Example 17

### 2-(2-(4-Methyl-3-(trifluoromethyl)phenyl)thiazol-4-yl)acetic acid

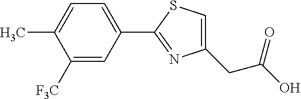

Example 17 was prepared from 4-methyl-3-trifluoromethyl-benzonitrile according to the synthesis described in example 8. The methyl ester was saponified as described in the synthesis of example 2. The product was recrystallized from EtOH/H₂O.

### Example 18

### 2-(2-(4-Fluoro-3-(trifluoromethyl)phenyl)thiazol-4-yl)acetic acid

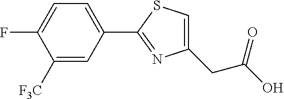

Example 18 was prepared from 4-fluoro-3-trifluoromethyl-benzonitrile according to the synthesis described in example 8. The methyl ester was saponified as described in the synthesis of example 2. The product was recrystallized from EtOH/H₂O.

### Example 19

### 2-(2-(3-Fluoro-5-(trifluoromethyl)phenyl)oxazol-4-yl)acetic acid

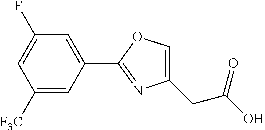

Example 19 was prepared from 3-fluoro-5-trifluoromethyl-benzamide (75 mg, 0.36 mmol) and methyl-4-chloroacetoacetate (1.26 μL, 1.09 mmol) according to the synthesis described in example 4. The methyl ester was saponified as described in the synthesis of example 2. The product was recrystallized from EtOH/H₂O.

### Example 20

### 2-(2-(3-Fluoro-5-(trifluoromethyl)phenyl)thiazol-4-yl)acetic acid

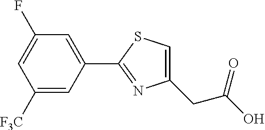

Example 20 was prepared from 3-fluoro-5-trifluoromethyl-benzonitrile according to the synthesis described in example 8. The methyl ester was saponified as described in the synthesis of example 2. The product was recrystallized from EtOH/H₂O.

### Example 21

### 2-(2-(2-Chloro-5-(trifluoromethyl)phenyl)thiazol-4-yl)acetic acid

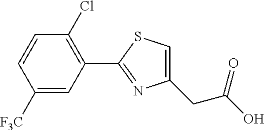

Example 21 was prepared from 2-chloro-5-trifluoromethyl-benzonitrile according to the synthesis described in example 8. The methyl ester was saponified as described in the synthesis of example 2. The product was recrystallized from EtOH/H₂O.

### Example 22

### 2-(2-(2-Chloro-3-(trifluoromethyl)phenyl)oxazol-4-yl)acetic acid

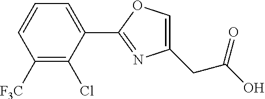

Example 22 was prepared from 2-chloro-3-trifluoromethyl-benzoic acid according to the synthesis described in example 15. The organic layer residue was dissolved in DMSO and the product purified from the reaction mixture via preparative HPLC.

### Example 23

### 2-(2-(4-Methoxy-3-(trifluoromethyl)phenyl)oxazol-4-yl)acetic acid

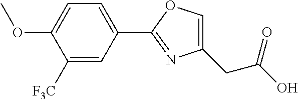

Example 23 was prepared from 4-methoxy-3-trifluoromethyl-benzamide (100 mg, 0.5 mmol) and methyl-4-chloroacetoacetate (226 mg, 1.5 mmol) according to the synthesis described in example 4. The methyl ester was saponified as described in the synthesis of example 2. The product was recrystallized from EtOH/H₂O.

### Example 24

### 2-(2-(4-Fluoro-3-(trifluoromethyl)phenyl)oxazol-4-yl)acetic acid

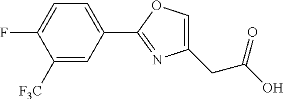

Example 24 was prepared from 4-fluoro-3-trifluoromethyl-benzamide (103 mg, 0.5 mmol) and methyl-4-chloroacetoacetate (226 mg, 1.5 mmol) according to the synthesis described in example 4. The methyl ester was saponified as described in the synthesis of example 2. The product was recrystallized from EtOH/H₂O.

### Example 25

### 2-(3-(4-Chloro-3-(trifluoromethyl)phenyl)isoxazol-5-yl)acetic acid

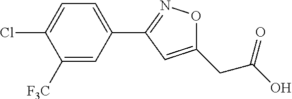

To a sealed vial was added 4-chloro-3-trifluoromethyl-benzaldehyde (1 g, 4.79 mmol), hydroxylamine hydrochloride (666 mg, 9.59 mmol), potassium carbonate (1.3 g, 9.59 mmol), and EtOH (8 mL). The reaction was stirred for 4 hr at ambient temperature, then diluted with H₂O and the pH adjusted to pH ˜6-7 with 10% citric acid. The aqueous layer was then extracted with EtOAc, washed with H₂O, dried over sodium sulfate and concentrated under reduced pressure. The resulting solid oxime was filtered, washed with Hexanes:CH₂Cl₂ (10:1) and used in the next step without further purification.

To a stirring solution of the oxime (400 mg, 1.79 mmol) in DMF (1 mL) at 0° C. was added N-chlorosuccinimide (239 mg, 1.79 mmol). The reaction was stirred at 50° C. for 45 min, then cooled to ambient temperature and diluted with H₂O. The aqueous layer was extracted with EtOAc, washed with H₂O (3×) and brine, dried over sodium sulfate, and concentrated under reduced pressure. The crude product was used in the next step without further purification.

To a stirring solution of the oxime chloride (338 mg, 1.31 mmol) and 3-butyn-1-ol (99 μL, 1.31 mmol) in CH₂Cl₂ at 0° C. was added triethylamine (183 μL, 1.31 mmol). The reaction was stirred at ambient temperature overnight. Upon completion, the reaction mixture was diluted with H₂O and extracted with EtOAC. The organics were combined, washed with H₂O, dried over sodium sulfate, and concentrated under reduced pressure. To a solution of the resulting alcohol (175 mg, 0.343 mmol) in CH₂Cl₂ (4 mL), was then added Dess-Martin Periodinane (174 mg, 0.411) at 0° C. The reaction was stirred at ambient temperature for 2 hr, after which it was quenched with saturated Na₂S₂O₃ and saturated NaHCO₃ and stirred at ambient temperature for an additional 5 min The layers were separated and the aqueous layer washed with CH₂Cl₂ (3×). The organic layers were combined and washed with brine, dried over sodium sulfate, and concentrated under reduced pressure.

A solution of sodium chlorite (195 mg, 2.15 mmol) in H₂O (0.3 mL) was then added to a mixture of the previously obtained aldehyde (78 mg, 0.269 mmol), sodium dihydrogen phosphate monohydrate (297 mg, 2.15 mmol) and 2-methyl-2-butene (0.5 mL) in THF (1 mL) at 0° C. The reaction mixture was stirred overnight at ambient temperature, then quenched with 10% Citric acid and extracted with EtOAc (3×). Organic layers were combined, washed with H₂O and brine, dried over sodium sulfate and concentrated under reduced pressure to give the desired isoxazole. The organic layer residue was dissolved in DMSO and the product purified from the reaction mixture via preparative HPLC.

### Example 26

### 2-(2-(4-Methoxy-3-(trifluoromethyl)phenyl)thiazol-4-yl)acetic acid

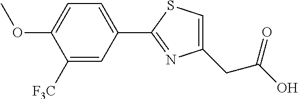

To a vial was added 4-methoxy-3-trifluoromethyl-benzamide (219 mg, 1.0 mmol) and Lawesson's Reagent (425 mg, 1.05 mmol) and THF (5 mL). The reaction was stirred in the microwave at 90° C. for 15 minutes, followed by standard aqueous work-up. The solvents were removed under reduced pressure and the compound purified via silica gel chromatography (5%-50% EtOAc/Hexanes) to give the desired product. The thioamide (60 mg, 0.25 mmol) was reacted with methyl-4-chloroacetoacetate (38 mg, 0.25 mmol) according to the synthesis described in example 1. The methyl ester was saponified as described in the synthesis of example 2. The product was recrystallized from EtOH/H₂O.

### Example 27

### 2-(2-(2-Chloro-3-(trifluoromethyl)phenyl)thiazol-4-yl)acetic acid

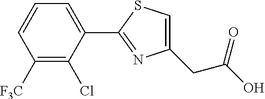

Example 27 was prepared from 2-chloro-3-trifluoromethyl-benzoic acid according to the syntheses described in examples 15 and 26. The product was recrystallized from EtOH/H₂O.

### Example 28

### 2-(2-(3-Bromo-5-(trifluoromethyl)phenyl)thiazol-4-yl)acetic acid

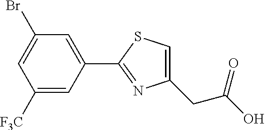

Example 28 was prepared from 3-bromo-5-trifluoromethyl-benzamide according to the synthesis described in example 26. The organic layer residue was dissolved in DMSO and the product purified from the reaction mixture via preparative HPLC.

### Example 29

### 2-(2-(3,4-Difluoro-5-(trifluoromethyl)phenyl)oxazol-4-yl)acetic acid

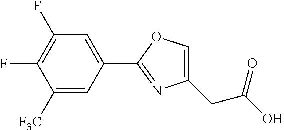

Example 29 was prepared from 3,4-difluoro-5-trifluoromethyl-benzamide (225 mg, 1.0 mmol) and methyl-4-chloroacetoacetate (452 mg, 3.0 mmol) according to the synthesis described in example 4. The methyl ester was saponified as described in the synthesis of example 2. The product was recrystallized from EtOH/H₂O.

### Example 30

### 2-(2-(3,4-Difluoro-5-(trifluoromethyl)phenyl)thiazol-4-yl)acetic acid

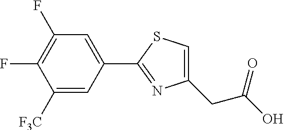

Example 30 was prepared from 3,4-difluoro-5-trifluoromethyl-benzoic acid according to the syntheses described in examples 15 and 26. The product was recrystallized from EtOH/H₂O.

### Example 31

### Methyl 2-(2-(3-(difluoromethyl)-5-(trifluoromethyl)phenyl)thiazol-4-yl)acetate

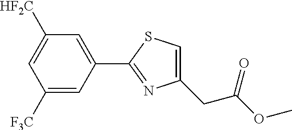

To a sealed vial was added [2-(3-bromo-5-trifluoromethyl-phenyl)-thiazol-4-yl]-acetic acid methyl ester (1.2 g, 3.16 mmol), styrene (542 μL, 4.73 mmol), Pd₂(dba)₃ (43 mg, 0.047 mmol), [(t-Bu)₃PH]BF₄ (28 mg, 0.095 mmol) N,N-dicyclohexylmethylamine (738 μL, 3.48 mmol) and dioxane (5 mL). The reaction was stirred at 95° C. for 2 hr, cooled to ambient temperature, filtered through celite, and washed with EtOAc. The solvent was removed under reduced pressure and the reaction purified via silica gel chromatography. The resulting alkyne was dissolved in CH₂Cl₂ and stirred under ozone for 15 min, then quenched with dimethyl sulfide and saturated sodium sulfite (aq) and stirred overnight at ambient temperature. The organic layers were separated and the aqueous layer is extracted with CH₂Cl₂ (3×). The organics were combined and washed with H₂O and brine, dried over MgSO₄ and concentrated under reduced pressure. The reaction was purified via silica gel chromatography (3:1 Hexanes/EtOAc) to give [2-(3-formyl-5-trifluoromethyl-phenyl)-thiazol-4-yl]acetic acid methyl ester.

DAST was added drop-wise to a solution of the aldehyde in CH₂Cl₂ at 0° C. under argon with stirring. MeOH was added, and the reaction mixture stirred at ambient temperature for 2 hr, then quenched with sat NaHCO₃ and extracted with EtOAc. The organic layers were combined, washed with saturated NaHCO₃, dried over MgSO₄, and the solvent removed under reduced pressure. The organic layer residue was dissolved in DMSO and the product purified from the reaction mixture via preparative HPLC.

### Example 32

### 2-(2-(3-(Difluoromethyl)-5-(trifluoromethyl)phenyl)thiazol-4-yl)acetic acid

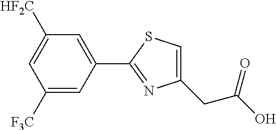

Example 32 was prepared from the methyl ester (example 31) according to the synthesis of example 2. The organic layer residue was dissolved in DMSO and the product purified from the reaction mixture via preparative HPLC.

### Example 33

### 4-(3,5-Bis-trifluoromethyl-phenyl)-thiazole-2-carboxylic acid

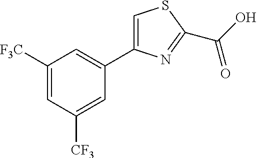

Example 33 was first prepared from 1-(3,5-bis-trifluoromethyl-phenyl)-2-bromo-ethanone (1 g, 2.9 mmol) and 2,2-dimethyl-propionic acid thiocarbamoylmethyl ester (508 mg, 2.9 mmol) according to the synthesis of Example 1. The resulting pivolate ester (1.19 g, 2.9 mmol) was refluxed in 3N HCl in dioxane (7 mL) and dioxane (3 mL) for 20 hrs. Upon completion, the reaction was cooled to ambient temperature, diluted with H₂O and the aqueous layer extracted with EtOAc (3×). The organic layers were combined, washed with H₂O and brine, then dried over MgSO₄ and concentrated. The resulting alcohol (850 mg, 2.6 mmol) was then reacted with Dess-Martin Periodinane (1.3 g, 3.1 mmol) and CH₂Cl₂ at ambient temperature for 1 hr. The reaction was quenched with sat. Na₂S₂O₃ and sat. NaHCO₃, and stirred at ambient temperature for an additional 5 min The aqueous layer was extracted with CH₂Cl₂ (3×), and the organics dried over MgSO₄ and concentrated.

The resulting aldehyde (70 mg, 0.22 mmol) was oxidized using 2-methyl-2-butene, NaClO₂, KH₂PO₄, t-BuOH, THF, and H₂O according to the synthesis described in example 25, step 5. The final acid was dissolved in DMSO and the product purified from the reaction mixture via preparative HPLC.

### Example 34

### 5-[2-(3,5-Bis-trifluoromethyl-phenyl)-thiazol-4-ylmethyl]-2H-tetrazole

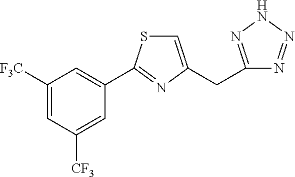

Example 34 was first prepared from 3,5-bis-trifluoromethyl-thiobenzamide (273 mg, 1.0 mmol) and 1,3-dichloroacetone (140 mg, 1.1 mmol) according to the synthesis of example 1. The resulting α-chloro thiazole was purified via silica gel chromatography (0%-40% EtOAc/Hexanes). To a sealed vial was added the chloride (345 mg, 1.0 mmol), KCN (195 mg, 3.0 mmol), K₂CO₃ (10%) and DMSO (5 mL). The reaction was stirred overnight at ambient temperature, after which it was poured over H₂O and extracted with EtOAc (3×). The organics were combined, washed with H₂O and brine, dried over MgSO₄ and concentrated. The product was then purified via silica gel chromatography (0%-40% EtOAc/Hexanes). The resulting nitrile (34 mg, 0.1 mmol) was then dissolved in DMF (1 mL). Sodium azide (33 mg, 0.5 mmol) and ammonium chloride (27 mg, 0.5 mmol) were added and the mixture was stirred at 140° C. overnight in a sealed reaction vessel. Upon completion, the reaction was cooled to ambient temperature, and the product purified from the reaction mixture via preparative HPLC.

### Example 35

### 5-[2-(4-Chloro-3-trifluoromethyl-phenyl)-thiazol-4-ylmethyl]-2H-tetrazole

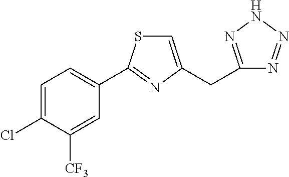

Example 35 was prepared from 4-chloro-3-trifluoromethyl-thiobenzamide and 1,3-dichloroacetone according to the synthesis of example 34. The organic layer residue was dissolved in DMSO, and the product purified from the reaction mixture via preparative HPLC.

### Example 36

### 2-(2-(2-Chloro-5-(trifluoromethyl)phenyl)oxazol-4-yl)acetic acid

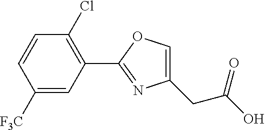

2-chloro-5-trifluoromethyl-benzonitrile (550 mg 2.68 mmol) was dissolved in 4N NaOH (3 mL) and THF (6 mL) and heated at 60° C. overnight. Upon completion, the reaction was cooled to ambient temperature and diluted with 10% citric acid. The aqueous layer was extracted with EtOAc, washed with 10% citric acid (2×) and brine, dried over MgSO₄, concentrated under reduced pressure and purified via silica gel chromatography (3:2 Hexanes/EtOAc). The resulting 2-chloro-5-trifluoromethyl-benzamide (100 mg, 0.447 mmol) was reacted with methyl-4-chloroacetoacetate (300 mg, 1.34 mmol) according to the synthesis described in example 4. The methyl ester was saponified as described in the synthesis of example 2. The organic layer residue was dissolved in DMSO, and the product purified from the reaction mixture via preparative HPLC.

### Example 37

### 2-(2-(3-Bromo-5-(trifluoromethyl)phenyl)oxazol-4-yl)acetic acid

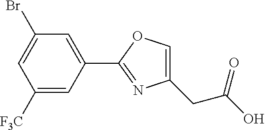

Example 37 was prepared from 3-bromo-5-trifluoromethyl-benzamide according to the synthesis described in example 4. The methyl ester was saponified as described in the synthesis of example 2. The organic layer residue was dissolved in DMSO and the product purified from the reaction mixture via preparative HPLC.

### Example 38

### Testing for the Efficacy of Compounds to Modulate RBP4

To determine the binding affinity of test compounds for RBP4, a homogeneous time resolved fluorescence (HTRF) assay was developed. The assay was a competition experiment measuring the interaction of RBP4 with the known RBP4 ligand retinol. Cy5-labeled retinol was incubated with 10 nM biotin-labeled RBP4 protein and streptavidin-Europium cryptate (1 nM). Test or control compounds were added to the reaction mixture and incubated at room temperature for 30 min. The HTRF signal was determined by monitoring the emission at 665 nM and 620 nM. The ratio between the 665 nM signal and the 620 nM signal was used to determine the binding of Cy5-retinol to RBP4. The ability of test or control compounds to displace Cy5-retinol from RBP4 was used to determine their potency for RBP4.

The exemplified compounds of the invention are summarized in Table 1.

TABLE 1

| | | Physical Data | Retinol |
|---|---|---|---|
| | | ¹H NMR 400 MHz (DMSO_{d6}-CDCl₃) | Displacement |
| Example | Structure | and/or MS (m/z) | IC₅₀ (μM) |
| 1 | 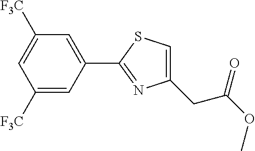 | ¹H NMR (400 MHz, MeOD-_{d4}): δ 8.38 (s, 2H), 7.96 (s, 1H), 7.49 (s, 1H), 3.86 (s, 2H), 3.65 (s, 3H). MS: (ES⁺) 369 m/z (M + 1)⁺ C₁₄H₉F₆NO₂S | 2.43 |
| 2 | 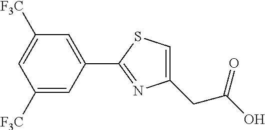 | ¹H NMR (400 MHz, MeOD-_{d4}): δ 8.53 (s, 2H), 8.01 (s, 1H), 7.62 (s, 1H), 3.95 (s, 2H). MS: (ES⁺) 355 m/z (M + 1)⁺ C₁₃H₇F₆NO₂S | 0.085 |
| 3 | 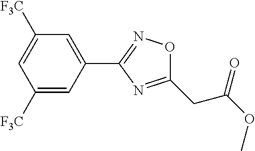 | ¹H NMR (400 MHz, DMSO-_{d6}): δ 8.55 (s, 2H), 8.46 (s, 1H), 2.72 (s, 2H), 2.37 (s, 3H). MS: (ES⁺) 354 m/z (M + 1)⁺ C₁₃H₈F₆N₂O₃ | 3.44 |
| 4 | 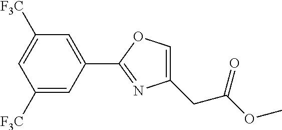 | ¹H NMR (400 MHz, MeOD-_{d4}): δ 8.38 (s, 2H), 7.95 (s, 1H), 7.60 (s, 1H), 3.40 (s, 2H), 3.28 (s, 3H). MS: (ES+) 353 m/z (M + 1)+ C₁₄H₉F₆NO₃ | 2.85 |
| 5 | 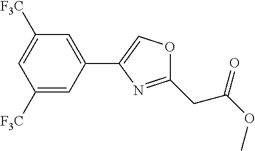 | ¹H NMR (400 MHz, MeOD-_{d4}): δ 8.32 (s, 1H), 8.18 (s, 2H), 7.65 (2, 1H), 3.75 (s, 2H), 3.45 (s, 3H). MS: (ES⁺) 353 m/z (M + 1)⁺ C₁₄H₉F₆NO₃ | 0.924 |
| 6 | 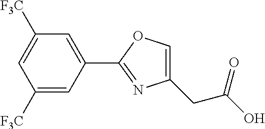 | ¹H NMR (400 MHz, MeOD-_{d4}): δ 8.45 (s, 2H), 8.01 (s, 1H), 7.92 (s, 1H), 3.61 (s, 2H) MS: (ES⁺) 339 m/z (M + 1)⁺ C₁₃H₇F₆NO₃ | 0.093 |
| 7 | 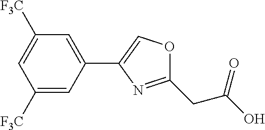 | ¹H NMR (400 MHz, MeOD-_{d4}): δ 8.48 (s, 1H), 8.26 (s, 2H), 7.80 (2, 1H), 3.87 (s, 2H). MS: (ES⁺) 339 m/z (M + 1)⁺ C₁₃H₇F₆NO₃ | 0.031 |
| 8 | 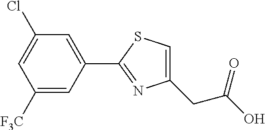 | ¹H NMR (400 MHz, MeOD-_{d4}): δ 8.22 (s, 1H), 8.16 (s, 1H), 7.79 (s, 1H), 7.54 (s, 1H), 3.90 (s, 2H). MS: (ES⁺) 320 m/z (M + 1)⁺ C₁₂H₇ClF₃NO₂S | 0.088 |
| 9 | 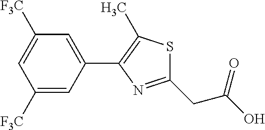 | ¹H NMR (400 MHz, , MeOD-_{d4}): δ 8.13 (s, 2H), 7.86 (s, 1H), 3.96 (s, 1H), 2.51 (s, 3H). MS: (ES⁺) 369 m/z (M + 1)⁺ C₁₄H₉F₆NO₂S | 0.232 |
| 10 | 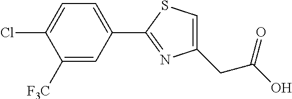 | ¹H NMR (400 MHz, , MeOD-_{d4}): δ 8.24 (d, J = 2 Hz, 1H), 8.03 (dd, J = 8.4, 2 Hz, 1H), 7.62 (d, J = 8.4 Hz, 1H), 7.40 (s, 1H), 3.78 (s, 2H). MS: (ES⁺) 320 m/z (M + 1)⁺ C₁₂H₇ClF₃NO₂S | 0.063 |
| 11 | 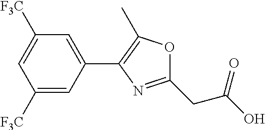 | ¹H NMR (400 MHz, , MeOD-_{d4}): δ 8.42 (s, 2H), 8.01 (s, 1H), 3.90 (s, 1H), 2.43 (s, 3H). MS: (ES⁺) 353 m/z (M + 1)⁺ C₄H₉F₆NO₃ | 1.59 |
| 12 | 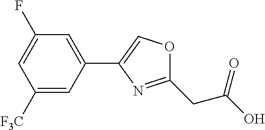 | ¹H NMR (400 MHz, , MeOD-_{d4}): δ 8.46 (s, 2H), 7.94 (s, 2H), 3.95 (s, 2H). MS: (ES⁺) 289 m/z (M + 1)⁺ C₁₂H₇F₄NO₃ | 0.073 |
| 13 | 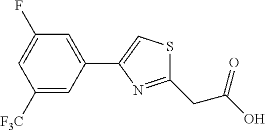 | ¹H NMR (400 MHz, , MeOD-_{d4}): δ 8.18 (s, 1H), 8.04 (s, 1H), 7.94 (d, J = 10.0 Hz, 1H), 7.39 (d, J = 8.4 Hz, 1H), 4.14 (s, 1H). MS: (ES⁺) 305 m/z (M + 1)⁺ C₁₂H₇F₄NO₂S | 0.091 |
| 14 | 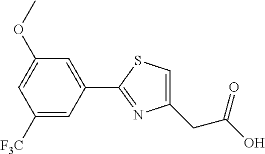 | ¹H NMR (400 MHz, , MeOD-_{d4}): δ 7.77 (s, 1H), 7.73 (s, 1H), 7.49 (s, 1H), 7.27 (s, 1H), 3.94 (s, 3H), 3.89 (s, 2H). MS: (ES⁺) 317 m/z (M + 1)⁺ C₁₃H₁₀F₃NO₃S | 0.088 |
| 15 | 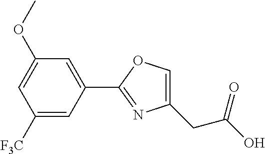 | ¹H NMR (400 MHz, , MeOD-_{d4}): δ 7.84 (s, 1H), 7.76 (s, 1H), 7.70 (s, 1H), 7.22 (s, 1H), 3.84 (s, 3H), 3.81 (s, 2H). MS: (ES⁺) 301 m/z (M + 1)⁺ C₁₃H₁₀F₃NO₄ | 0.248 |
| 16 | 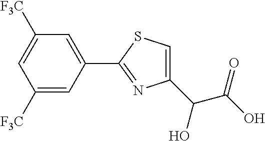 | ¹H NMR (400 MHz, , MeOD-_{d4}): δ 8.42 (s, 2H), 8.08 (s, 1H), 7.81 (s, 1H), 4.83 (s, 1H) MS: (ES⁺) 371 m/z (M + 1)⁺ C₁₃H₇F₆NO₃S | 0.141 |
| 17 | 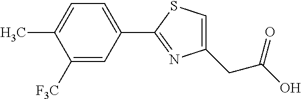 | ¹H NMR (400 MHz, , MeOD-_{d4}): δ 8.11 (s, 1H), 7.93 (d, J = 8.0, 1.5 Hz, 1H), 7.39 (d, J = 8.4 Hz, 1H), 7.34 (s, 1H), 3.77 (s, 2H), 2.43 (s, 3H). MS: (ES⁺) 301 m/z (M + 1)⁺ C₁₃H₁₀F₃NO₂S | 0.086 |
| 18 | 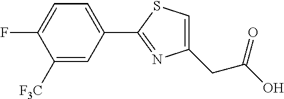 | ¹H NMR (400 MHz, , MeOD-_{d4}): δ 8.28 (dd, J = 6.8, 2.0 Hz, 1H, 8.25- 8.21 (m, 1H), 7.49-7.45 (m, 2H), 3.88 (s, 2H). MS: (ES⁺) 305 m/z (M + 1)⁺ C₁₂H₇F₄NO₂S | 0.44 |
| 19 | 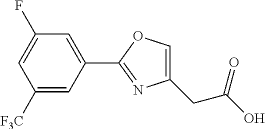 | ¹H NMR (400 MHz, , MeOD-_{d4}): δ 8.13 (s, 1H), 7.99 (d, J = 12.4 Hz, 2H), 7.62 (d, J = 8.0 Hz, 1H), 3.70 (s, 2H). MS: (ES⁺) 289 m/z (M + 1)⁺ C₁₂H₇F₄NO₃ | 0.658 |
| 20 | 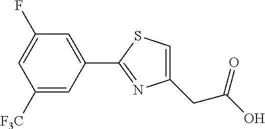 | ¹H NMR (400 MHz, , MeOD-_{d4}): δ 8.05 (d, J = 3.2 Hz, 1H), 7.83 (d, J = 9.6 Hz, 2H), 7.62 (d, J = 8.0 Hz, 1H), 2.99 (s, 2H). MS: (ES⁺) 305 m/z (M + 1)⁺ C₁₂H₇F₄NO₂S | 0.168 |
| 21 | 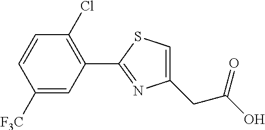 | ¹H NMR (400 MHz, , MeOD-_{d4}): δ 8.53 (s, 1H), 7.73 (q, J = 11.2, 8.4 Hz, 2H), 7.64 (s, 1H), 3.93 (s, 2H). MS: (ES⁺) 320 m/z (M + 1)⁺ C₁₂H₇ClF₃NO₂S | 1.05 |
| 22 | 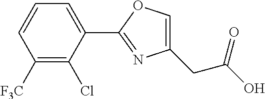 | ¹H NMR (400 MHz, , MeOD-_{d4}) δ 8.03 (d, J = 8.0 Hz, 1H), 7.93 (s, 1H), 7.85 (d, J = 7.6 Hz), 7.53 (t, J = 8.0 Hz, 1H), 3.62 (s, 2H). MS: (ES⁺) 305 m/z (M + 1)⁺ C₁₂H₇ClF₃NO₃ | 1.66 |
| 23 | 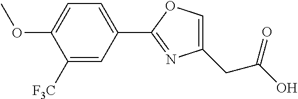 | ¹H NMR (400 MHz, , MeOD-_{d4}): δ 8.10 (t, J = 3.6 Hz, 2H), 7.77 (s, 1H), 7.22 (d, J = 9.2 Hz, 1H), 3.88 (s, 3H), 3.56 (s, 2H). MS: (ES⁺) 301 m/z (M + 1)⁺ C₁₃H₁₀F₃NO₄ | 0.175 |
| 24 | 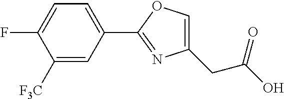 | ¹H NMR (400 MHz, , MeOD-_{d4}): δ 8.21-8.18 (m, 2H), 7.84 (s, 1H), 7.40 (t, J = 10 Hz, 1H), 3.58 (s, 2H). MS: (ES⁺) 289 m/z (M + 1)⁺ C₁₂H₇F₄NO₃ | 0.721 |
| 25 | 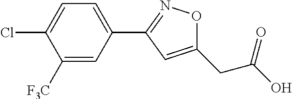 | ¹H NMR (400 MHz, , MeOD-_{d4}): δ 8.23 (s, 1H), 8.07 (d, J = 8.4 Hz, 1H), 7.74 (d, J = 8.4 Hz, 1H), 6.89 (s, 1H), 3.96 (s, 2H). MS: (ES⁺) 305 m/z (M + 1)⁺ C₁₂H₇ClF₃NO₃ | 0.285 |
| 26 | 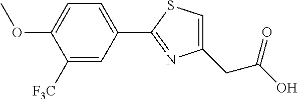 | ¹H NMR (400 MHz, , MeOD-_{d4}): δ 8.15 (d, J = 2.4 Hz, 1H), 8.10 (dd, J = 8.4, 2.4 Hz, 1H), 7.37 (s, 1H), 7.28 (d, J = 8.8 Hz, 1H), 3.97 (s, 3H), 3.86 (s, 2H). MS: (ES⁺) 317 m/z (M + 1)⁺ C₁₃H₁₀F₃NO₃S | 0.193 |
| 27 | 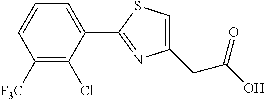 | ¹H NMR (400 MHz, , MeOD-_{d4}): δ 8.20 (d, J = 8.0 Hz, 1H), 8.29 (dd, J = 8.0, 1.2 Hz, 1H), 7.54 (s, 1H), 7.49 (t, J = 8.0 Hz, 1H), 3.81 (s, 2H). MS: (ES⁺) 320 m/z (M + 1)⁺ C₁₂H₇ClF₃NO₂S | 2.385 |
| 28 | 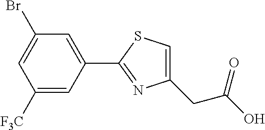 | ¹H NMR (400 MHz, , MeOD-_{d4}): δ 8.34 (s, 1H), 8.18 (s, 1H), 7.91 (s, 1H), 7.53 (s, 1H), 3.89 (s, 2H). MS: (ES⁺) 364 m/z (M + 1)⁺ C₁₂H₇BrF₃NO₂S | 0.05 |
| 29 | 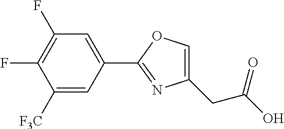 | ¹H NMR (400 MHz, , MeOD-_{d4}): δ 8.12-8.05 (m, 1H), 8.02-8.01 (m, 1H), 7.86 (s, 1H), 3.58 (s, 2H). MS: (ES⁺) 307 m/z (M + 1)⁺ C₁₂H₆F₅NO₃ | 0.263 |
| 30 | 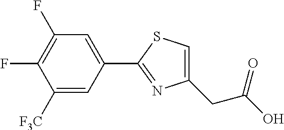 | ¹H NMR (400 MHz, , MeOD-_{d4}): δ 8.12-8.07 (m, 1H), 7.97 (d, J = 5.6 Hz, 1H), 7.42 (s, 1H), 3.78 (s, 2H). MS: (ES⁺) 323 m/z (M + 1)⁺ C₁₂H₆F₅NO₂S | 0.086 |
| 31 | 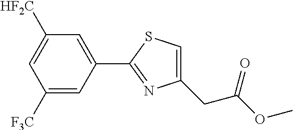 | ¹H NMR (400 MHz, , MeOD-_{d4}): δ 8.37 (d, J = 6.4 Hz, 2H), 7.94 (s, 1H), 7.57 (s, 1H), 6.98 (t, J = 55.6 Hz, 1H), 3.96 (s, 2H), 3.75 (s, 3H). MS: (ES⁺) 351 m/z (M + 1)⁺ C₁₄H₁₀F₅NO₂S | |
| 32 | 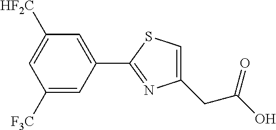 | ¹H NMR (400 MHz, , CD₃CN-_{d3}): δ 8.33 (s, 2H), 7.94 (s, 1H), 7.46 (s, 1H), 6.94 (t, J = 55.2 Hz, 1H), 3.87 (s, 2H). MS: (ES⁺) 337 m/z (M + 1)⁺ C₁₃H₈F₅NO₂S | 0.134 |
| 33 | 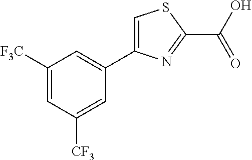 | ¹H NMR (400 MHz, , MeOD-_{d4}): δ 8.65 (s, 2H), 8.58 (s, 1H), 7.80 (s, 1H). MS: (ES⁺) 340 m/z (M + 1)⁺ C₁₂H₅F₆NO₂S | 1.06 |
| 34 | 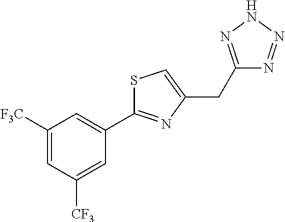 | ¹H NMR (400 MHz, , MeOD-_{d4}): δ 8.51 (s, 2H), 8.07 (s, 1H), 7.64 (s, 1H), 4.59 (s, 2H). MS: (ES⁺) 379 m/z (M + 1)⁺ C₁₃H₇F₆N₅S | 0.098 |
| 35 | 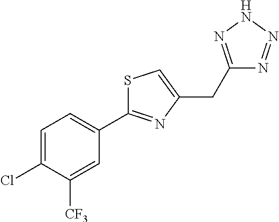 | ¹H NMR (400 MHz, , MeOD-_{d4}): δ 8.35 (s, 1H), 8.15 (d, J = 8.4 Hz, 1H), 7.73 (d, J = 8.4 Hz, 1H), 7.56 (s, 1H), 4.56 (s, 2H). MS: (ES⁺) 345 m/z (M + 1)⁺ C₁₂H₇ClF₃N₅S | 0.196 |
| 36 | 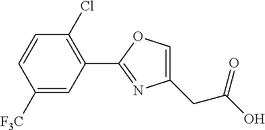 | ¹H NMR (400 MHz, , MeOD-_{d4}): δ 8.60 (s, 1H), 7.75 (q, J = 11.2, 8.4 Hz, 2H), 7.64 (s, 1H), 3.81 (s, 2H). MS: (ES⁺) 305 m/z (M + 1)⁺ C₁₂H₇ClF₃NO₃ | 8.82 |
| 37 | 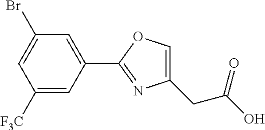 | ¹H NMR (400 MHz, , MeOD-_{d4}): δ 8.44 (s, 2H), 8.32 (s, 1H), 8.28 (s, 1H), 2.99 (s, 2H). MS: (ES⁺) 348 m/z (M + 1)⁺ C₁₂H₇BrF₃NO₃ | >10 |

Assays

For the development of a TR-FRET based RBP4-binding assay, the carboxy-group of retinoic acid (RA) was derivatized to attach a Cy5-fluorophor separated by a short PEG spacer resulting in RA-PEG-Cy5. To monitor RA-PEG-Cy5 binding to its carrier protein, RBP4 was biotinylated and preincubated with a streptavidin-europium chelate conjugate (SA-Eu). When RA-Cy5 binding to RBP4-biotin/SA-Eu complexes occurs, the excitation of the Eu-chelates with UV light around 330 nm leads to light emission at 620 nm, energy transfer to Cy5 and subsequent light emission by Cy5 at 665 nm. Therefore, light emission at 665 nm or, alternatively, the ratio of light emitted at 665 nm and 620 nm is a direct measure for RA-PEG-Cy5 binding to RBP4-Eu.

In order to identify compounds that not only displace retinol or retinoic acid from RBP4 but also disrupt the RBP4-TTR interaction, a secondary SPR based assay was developed. To this end, biotinylated TTR was loaded on a streptavidin coated biosensor chip and RBP4 solutions were passed over the immobilized TTR at varying concentrations in the presence of test compounds or DMSO alone.

Testing for the Efficacy of Compounds for the Treatment of Macular Degeneration

For pre-testing, all human patients will undergo a routine ophthalmologic examination including fluorescein angiography, measurement of visual acuity, electrophysiologic parameters and biochemical and rheologic parameters. Inclusion criteria are as follows: visual acuity between 20/160 and 20/32 in at least one eye and signs of AMD such as drusen, areolar atrophy, pigment clumping, pigment epithelium detachment, or subretinal neovascularization. Patients that are pregnant or actively breast-feeding children will be excluded from the study.

Two hundred human patients diagnosed with macular degeneration, or who have progressive formations of A2E, lipofuscin, or drusen in their eyes will be divided into a control group of about 100 patients and an experimental group of 100 patients. A compound of the invention will be administered to the experimental group on a daily basis. A placebo will be administered to the control group in the same regime as a compound of the invention is administered to the experimental group. Administration of a compound of the invention or placebo to a patient can be either orally or parenterally administered at amounts effective to inhibit the development or reoccurrence of macular degeneration. Effective dosage amounts are in the range of from about 1-4000 mg/m2 up to three times a day.

One method for measuring progression of macular degeneration in both control and experimental groups is the best corrected visual acuity as measured by Early Treatment Diabetic Retinopathy Study (ETDRS) charts (Lighthouse, Long Island, N.Y.) using line assessment and the forced choice method (Ferris et al., Am J Ophthalmol., 94:97-98 (1982)). Visual acuity is recorded in logMAIR. The change of one line on the ETDRS chart is equivalent to 0.1 logMAR. Further typical methods for measuring progression of macular degeneration in both control and experimental groups include use of visual field examinations, including but not limited to a Humphrey visual field examination, and measuring/monitoring the autofluorescence or absorption spectra of N-retinylidene-phosphatidylethanolamine, dihydro-N-retinylidene-N-retinyl-phosphatidylethanolamine, N-retinylidene-N-retinyl-phosphatidylethanolamine, dihydro-N-retinylidene-N-retinyl-ethanolamine, and/or N-retinylidene-phosphatidylethanolamine in the eye of the patient. Autofluorescence is measured using a variety of equipment, including but not limited to a confocal scanning laser ophthalmoscope. See Bindewald et al., Am. J. Ophthalmol., 137:556-8 (2004).

Additional methods for measuring progression of macular degeneration in both control and experimental groups include taking fundus photographs, observing changes in autofluorescence over time using a Heidelberg retina angiograph (or alternatively, techniques described in Hammer et al., Opthalmologe 2004 Apr. 7 [Epub ahead of patent), and taking fluorescein angiograms at baseline, three, six, nine and twelve months at follow-up visits. Documentation of morphologic changes include changes in (a) drusen size, character, and distribution; (b) development and progression of choroidal neovascularization; (c) other interval fundus changes or abnormalities; (d) reading speed and/or reading acuity; (e) scotoma size; or (f) the size and number of the geographic atrophy lesions. In addition, Amsler Grid Test and color testing are optionally administered.

To assess statistically visual improvement during drug administration, examiners use the ETDRS (LogMAR) chart and a standardized refraction and visual acuity protocol. Evaluation of the mean ETDRS (LogMAR) best corrected visual acuity (BCVA) from baseline through the available post-treatment interval visits can aid in determining statistical visual improvement. To assess the ANOVA (analysis of variance between groups) between the control and experimental group, the mean changes in ETDRS (LogMAR) visual acuity from baseline through the available post-treatment interval visits are compared using two-group ANOVA with repeated measures analysis with unstructured covariance using SAS/STAT Software (SAS Institutes mc, Cary, N.C.). Toxicity evaluation after the commencement of the study includes check ups every three months during the subsequent year, every four months the year after and subsequently every six months.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A compound having Formula (1) or (2): 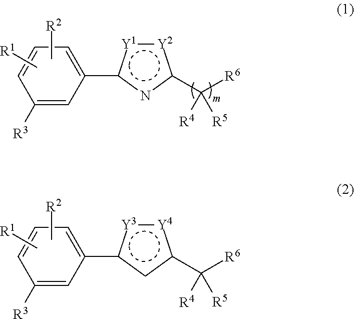
or a physiologically acceptable salt thereof;
wherein R¹ and R² are independently H, halogen, C₁₋₆ alkoxy, or a C₁₋₆ alkyl optionally substituted with halogen, provided R¹ and R² are not both H;
R³ is C₁₋₆ halogenated alkyl;
R⁴ and R⁵ are independently H, OH, C₁₋₆ alkyl, C₁₋₆ alkoxy or C₃₋₇ carbocyclic ring; or R⁴ and R⁵ together may form a 3-6 membered ring;
R⁶ is CO₂R⁷;
R⁷ is H or C₁₋₆ alkyl;
one of Y¹ and Y² is S or O and the other is CR⁸ wherein R⁸ is H or C₁₋₆ alkyl; alternatively, one of Y¹ and Y² is N and the other is O;
one of Y³ and Y⁴ is N and the other is O;
m is 0-1;
provided said compound does not have Formula (1-Q) or (1-R): 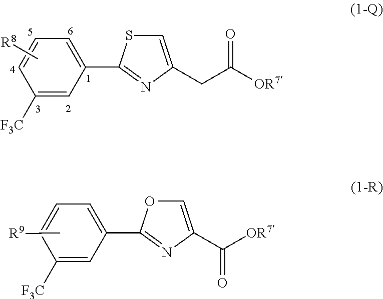
wherein R⁸ is halo at the 6-position of the phenyl ring;
R⁹ is halo; and
each R^{7′} is H or C₁₋₆ alkyl.

2. The compound of claim 1, wherein said compound is of Formula (1),
R¹ is halogen, C₁₋₆ alkoxy, or C₁₋₆ alkyl optionally substituted with halogen, and is at any position of the phenyl ring;
R² is H; and
R³, R⁴, R⁵, R⁶, Y¹, Y² and m are as defined in claim 1.

3. The compound of claim 1, wherein said compound is of Formula (1A): 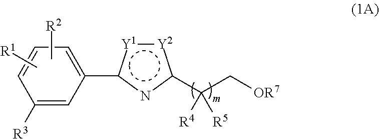
wherein R¹ and R² are halogen; and
R³, R⁴, R⁵, R⁷, Y¹, Y² and m are as defined in claim 1.

4. The compound of claim 1, wherein said compound is of Formula (1B): 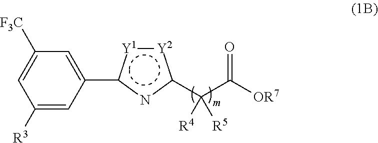
R³, R⁴, R⁵, R⁷, Y¹, Y² and m are as defined in claim 1.

5. The compound of claim 1, wherein Y¹ is S or O and Y² is CR⁸, and R⁸ is H or C₁₋₆ alkyl.

6. The compound of claim 1, wherein Y² is S or O and Y¹ is CR⁸, and R⁸ is H or C₁₋₆ alkyl.

7. The compound of claim 1, wherein one of Y¹ is N and the other is O.

8. The compound of claim 1, wherein m is 1.

9. The compound of claim 1, wherein said compound is of Formula (2); 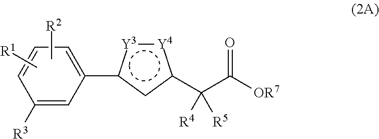
R¹, R², R³, R⁴, R⁵, R⁷, Y³ and Y⁴ are as defined in claim 1.

10. A compound having Formula (1) or (2): 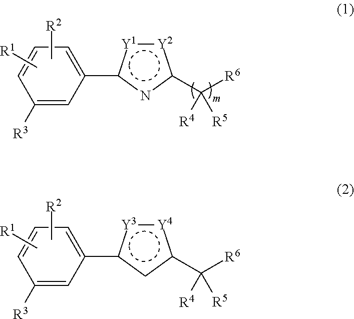
or a physiologically acceptable salt thereof;
wherein R¹ and R² are independently H, halogen, C₁₋₆ alkoxy, or a C₁₋₆ alkyl optionally substituted with halogen, provided R¹ and R² are not both H;
R³ is C₁₋₆ halogenated alkyl;
R⁴ and R⁵ are independently H, OH, C₁₋₆ alkyl, C₁₋₆ alkoxy or C₃₋₇ carbocyclic ring; or R⁴ and R⁵ together may form a 3-6 membered ring;
R⁶ is a carboxylic acid isostere;
one of Y¹ and Y² is S or O and the other is CR⁸ wherein R⁸ is H or C₁₋₆ alkyl; alternatively, one of Y¹ and Y² is N and the other is O;
one of Y³ and Y⁴ is N and the other is O;
m is 1;
provided said compound does not have Formula (1-Q) or (1-R): 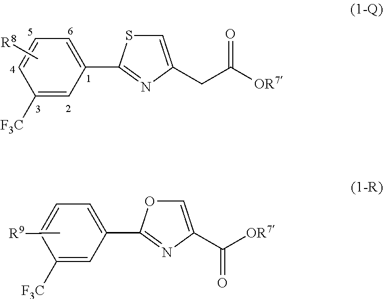
wherein R⁸ is halo at the 6-position of the phenyl ring;
R⁹ is halo; and
each R^{7′} is H or C₁₋₆ alkyl.

11. The compound of claim 10, wherein said carboxylic acid isostere is selected from the group consisting of

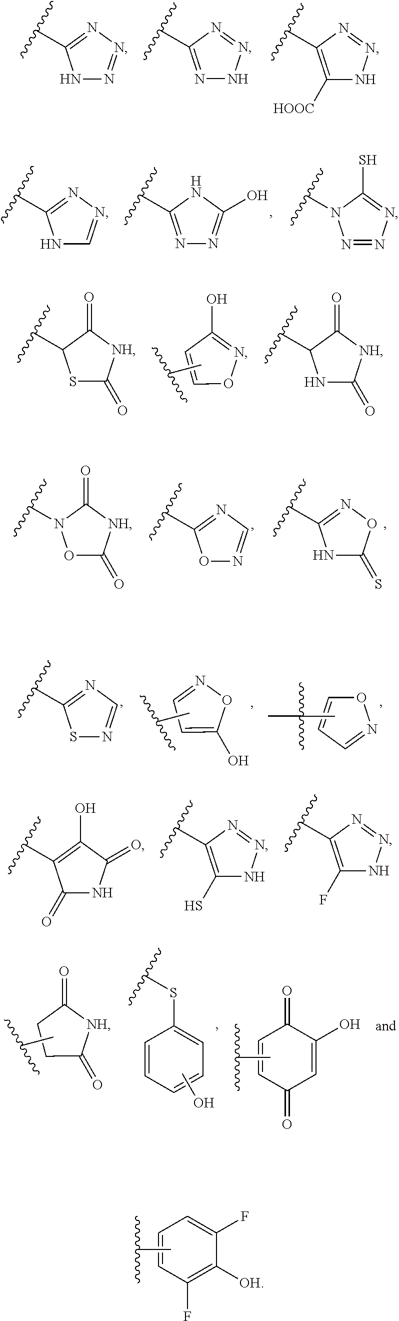

12. The compound of claim 1, wherein R³ is CF³.

13. The compound of claim 1, wherein R⁴ and R⁵ are H.

14. The compound of claim 1, wherein R⁴ is H and R⁵ is OH.

15. The compound of claim 1 wherein said compound is selected from the group

| Example | Structure |
|---|---|
| 1 | 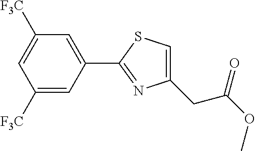 |
| 2 | 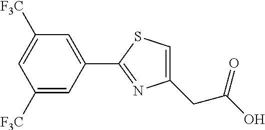 |
| 3 | 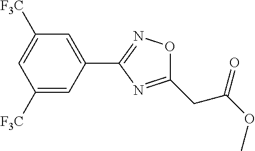 |
| 4 | 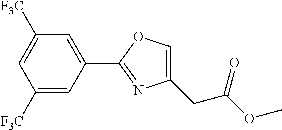 |
| 5 | 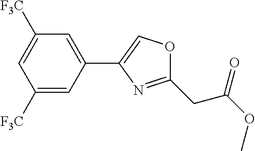 |
| 6 | 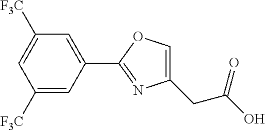 |
| 7 | 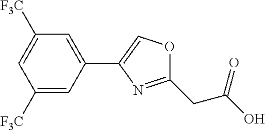 |
| 8 | 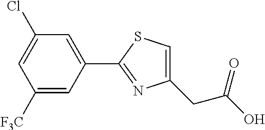 |
| 9 | 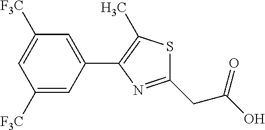 |
| 10 | 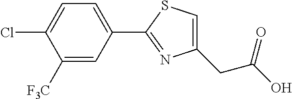 |
| 11 | 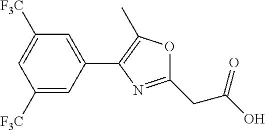 |
| 12 | 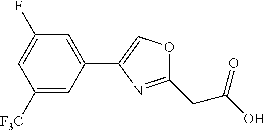 |
| 13 | 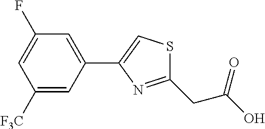 |
| 14 | 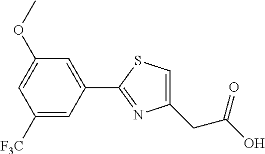 |
| 15 | 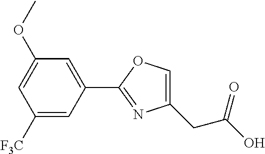 |
| 16 | 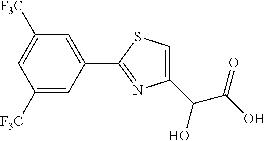 |
| 17 | 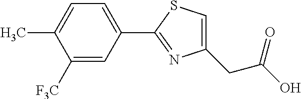 |
| 18 | 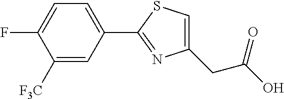 |
| 19 | 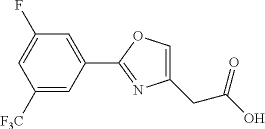 |
| 20 | 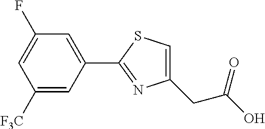 |
| 21 | 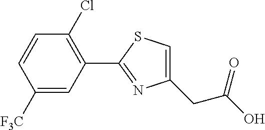 |
| 22 | 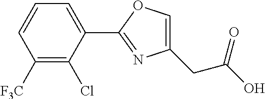 |
| 23 | 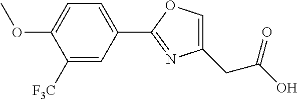 |
| 24 | 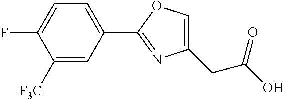 |
| 25 | 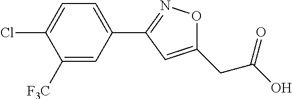 |
| 26 | 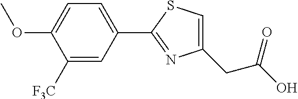 |
| 27 | 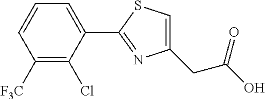 |
| 28 | 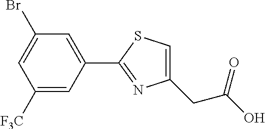 |
| 29 | 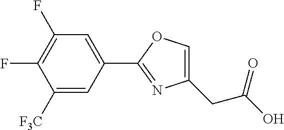 |
| 30 | 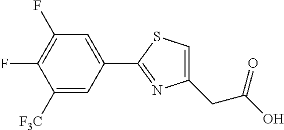 |
| 31 | 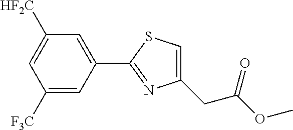 |
| 32 | 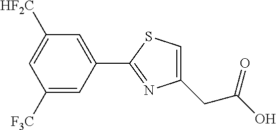 |
| 33 | 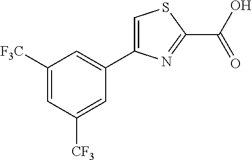 |
| 34 | 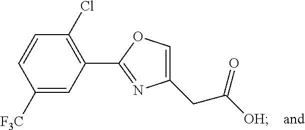 |
| 35 | 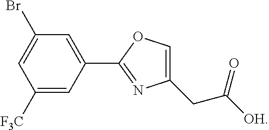 |

16. The compound of claim 10 wherein said compound is selected from the group

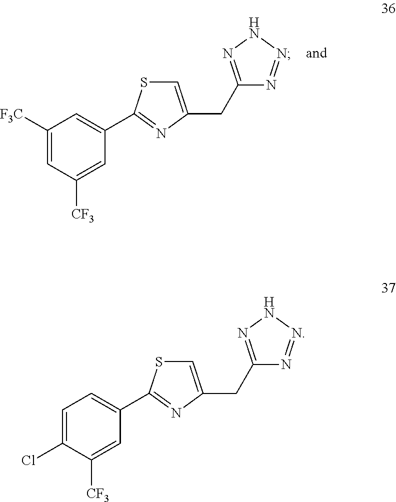

17. A pharmaceutical composition comprising a therapeutically effective amount of a compound of claim 1 and a physiologically acceptable carrier.

18. A method for inhibiting retinol binding to retinol binding protein 4 (RBP4) in a cell, comprising contacting the cell with an effective amount of a compound having Formula (1) or (2), 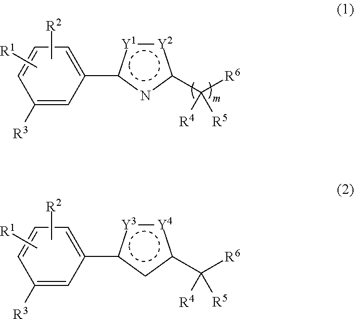
or a physiologically acceptable salt thereof;
wherein R¹ and R² are independently H, halogen, C₁₋₆ alkoxy, or a C₁₋₆ alkyl optionally substituted with halogen, provided R¹ and R² are not both H;
R³ is C₁₋₆ halogenated alkyl;
R⁴ and R⁵ are independently H, OH, C₁₋₆ alkyl, C₁₋₆ alkoxy or C₃₋₇ carbocyclic ring; or R⁴ and R⁵ together may form a 3-6 membered ring;
R⁶ is CO₂R⁷ or a carboxylic acid isostere other than 5,6-dihydro-1,4,2-dioxazinyl;
R⁷ is H or C₁₋₆ alkyl;
one of Y¹ and Y² is S or O and the other is CR⁸ wherein R⁸ is H or C₁₋₆ alkyl; alternatively, one of Y¹ and Y² is N and the other is O;
one of Y³ and Y⁴ is N and the other is O;
m is 0-1.

19. A method for treating a condition mediated by retinol binding to retinol binding protein 4 (RBP4) in a subject suffering therefrom, comprising administering to said subject an effective amount of a compound of Formula (1) or (2), 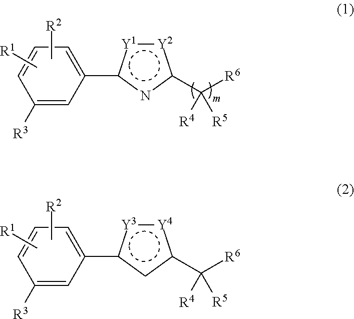
or a physiologically acceptable salt thereof;
wherein R¹ and R² are independently H, halogen, C₁₋₆ alkoxy, or a C₁₋₆ alkyl optionally substituted with halogen, provided R¹ and R² are not both H;
R³ is C₁₋₆ halogenated alkyl;
R⁴ and R⁵ are independently H, OH, C₁₋₆ alkyl, C₁₋₆ alkoxy or C₃₋₇ carbocyclic ring; or R⁴ and R⁵ together may form a 3-6 membered ring;
R⁶ is CO₂R⁷ or a carboxylic acid isostere other than 5,6-dihydro-1,4,2-dioxazinyl;
R⁷ is H or C₁₋₆ alkyl;
one of Y¹ and Y² is S or O and the other is CR⁸ wherein R⁸ is H or C₁₋₆ alkyl; alternatively, one of Y¹ and Y² is N and the other is O;
one of Y³ and Y⁴ is N and the other is O;
m is 0-1;
wherein said condition is macular degeneration or Stargardt's disease.

20. The method of claim 19, wherein said condition is age-related macular degeneration, atrophic age-related macular degeneration or Stargard's disease.

21-24. (canceled)
